(19) United States
(12) Patent Application Publication
(10) Pub. No.: US 2005/0020687 A1
(43) Pub. Date: Jan. 27, 2005

(54) Methods of treating inflammatory and viral disorders by administering cyclopentenone compounds

(75) Inventors: Maria Gabriella Santoro, Avellino (IT); Antonio Rossi, Colledimacine (IT); Giuliano Elia, Palestrina (IT); Enrico Garaci, Roma (IT); Carla Amici, Roma (IT)

Correspondence Address: JONES DAY, 222 EAST 41ST ST, NEW YORK, NY, 10017, US

(73) Assignee: Consiglio Nazionale Ricerche

(21) Appl. No.: 10/742,523

(22) Filed: Dec. 19, 2003

Related U.S. Application Data
(63) Continuation of application No. 09/533,399, filed on Mar. 22, 2000, now abandoned.
Application No. 09/533,399 is a continuation-in-part of application No. 09/202,553, filed on Dec. 16, 1998, now Pat. No. 6,180,681.
Application No. 09/533,399 is a continuation-in-part of application No. 09/446,731, filed on Dec. 23, 1999, now abandoned.

(30) Foreign Application Priority Data
Jun. 18, 1996 (IT) RM 96A000430
Jun. 13, 1996 (IT) RM 96A000867
Jan. 14, 1996 (IT) RM 97A000392
Jun. 18, 1997 (WO) PCT/EP97/03185
Dec. 11, 1997 (WO) PCT/EP97/06930
Jan. 14, 1999 (WO) PCT/EP98/04066

(51) Int. Cl. A61K 31/557

(52) U.S. Cl. 514/573

(57) ABSTRACT

The present invention relates to methods and compositions for the treatment and/or prevention of diseases and disorders in humans, including cancer, inflammatory diseases or disorders, and infectious diseases. In particular, the present invention relates to methods and compositions comprising the administration of a cyclopentenone prostaglandin or a derivative thereof, which induces cytoprotective responses and inhibits NF-κB activation. The present invention further relates to pharmaceutical compositions containing cyclopentenone prostaglandins for the treatment and/or prevention of viral infection, inflammation, and/or cancer in humans.

This application is a continuation-in-part of application Ser. No. 09/202,553, filed Dec. 16, 1998, application Ser. No. 09/319,743, filed Jun. 10, 1999, and application Ser. No. 09/446,731, filed Dec. 23, 1999, each of which is incorporated herein in its entirety.

### 1. FIELD OF THE INVENTION

The present invention relates to cyclopentenone compounds, including both natural and synthetic derivatives of cyclopentenone prostaglandins, having both cytoprotective inducing activities and nuclear factor-κB (NF-κB) inhibitory properties. The present invention further relates to methods for the treatment and prevention of virus infection comprising administering these cyclopentenone compounds to a human subject. The present invention also relates to cyclopentenone compounds and their derivatives for use as therapeutics in the treatment of inflammation, viral infection and cancer. The cyclopentenone compounds of the present invention preferably inhibit NF-κB activation and induce the expression or activity of heat shock proteins, such as hsp 70. The present invention further relates to pharmaceutical compositions for the treatment and prevention of viral infection, inflammation or cancer in humans.

Finally, the present invention encompasses assays that can be used to identify compounds having both cyctoprotective and NF-κB inhibitory properties.

### 2. BACKGROUND OF THE INVENTION

Despite large immunization programs, viral infections, especially influenza infections, remain a serious source of morbidity and mortality throughout the world and a significant cause of illness and death among people with immune-deficiency associated with aging or different clinical conditions (see, e.g., Hughes-Fulford et al., 1992, Antimicrob. Agents Chemother. 36: 2253-2258). Although antiviral chemotherapy with compounds such as amantadine and rimantadine have been shown to reduce the duration of symptoms of clinical infections (i.e., influenza infection), major side effects and the emergence of drug-resistant variants have been described (see, e.g. Couch et al., 1997, N. Engl. J. Med. 337: 927-928 and Hughes-Fulford et al., 1992, supra). New classes of antiviral agents designed to target particular viral proteins such as influenza neuraminidase are being developed. However, the ability of viruses to mutate the target proteins represents an obstacle for effective treatment with molecules which selectively inhibit the function of specific viral polypeptides.

Table of Contents

1. FIELD OF THE INVENTION . . . 1
2. BACKGROUND OF THE INVENTION . . . 1
   2.1 HEAT SHOCK PROTEINS . . . 2
   2.2 NF-κB . . . 3
3. SUMMARY OF THE INVENTION . . . 4
4. DESCRIPTION OF THE FIGURES . . . 5
5. DETAILED DESCRIPTION OF THE INVENTION . . . 9
   5.1. COMPOUNDS OF THE INVENTION . . . 11
   5.2. USES OF COMPOUNDS OF THE INVENTION . . . 13
   5.3. DEMONSTRATION OF THERAPEUTIC OR PROPHYLACTIC USES OF COMPOUNDS . . . 17
   5.4. THERAPEUTIC AND PHARMACEUTICAL COMPOSITIONS AND ADMINISTRATION THEREOF . . . 19
   5.5 SCREENING ASSAYS . . . 24
6. EXAMPLE: 2-CYCLOPENTEN-1-ONE INDUCES HSP70 EXPRESSION, INHIBITS VIRAL REPLICATION, AND INHIBITS NF-κB ACTIVATION . . . 26
7. EXAMPLE: 3,4-DICHLORO-ISO-COUMARINE INDUCES HSF ACTIVATION, INHIBITS VIRAL REPLICATION, AND INHIBITS NF-κB ACTIVATION . . . 29
8. EXAMPLE: Δ¹²-PROSTAGLANDIN J2 IS A POTENT INHIBITOR OF INFLUENZA A VIRUS REPLICATION IN VIVO . . . 33

One successful approach in combating viral infections appears to be the simultaneous use of two or more drugs that affect different targets during the virus life cycle. A group of prostaglandins (PG) and PG-derivatives containing an α,β-unsaturated carbonyl group in the cyclopentane ring (cyclopentenone PG, cyPG) have been shown in vitro to have the interesting ability to interfere with virus replication at multiple level (Santoro et al., 1997, Trends Microbiol. 5: 276-281). For example, prostaglandins of the A and J type (PGAs and PGJs) have been shown to inhibit the replication of a variety of RNA viruses, including paramyxoviruses, rhabdoviruses, rotaviruses and retroviruses in cultured cells (reviewed in Santoro et al., 1997, supra). The antiviral activity of cyclopentenonic prostaglandins has been attributed to the induction of heat shock protein (i.e., HSP70) synthesis and the inhibition of NF-κB activity (Amici et al., 1992, Proc. Natl. Acad. Sci. USA 89: 6227-6231 and Rossi et al., 1997, Proc. Natl. Acad. Sci. USA 94: 746-750). Cyclopentenonic prostaglandins induce HSP70 synthesis through the activation of the heat shock transcription factor (HSF) (Amici et al., 1992, supra). The induction of HSP70 synthesis has been suggested to be one of the molecular mechanisms used by cyclopentenonic prostaglandins to cause a selective and reversible block of the protein synthesis in in vitro infection models with single strand negatively polarized RNA viruses (Amici et al., J. Virol. 68, 6890-6899, 1994). Whereas there is an extensive literature on the antiviral activity of cyclopentenone prostanoids in in vitro experimental models, little is known on the therapeutic efficacy of these molecules in in vivo viral infection.

### 2.1 Heat Shock Proteins

Heat Shock Proteins (HSPs), also called stress proteins (1989, Proc. Natl. Acad. Sci. USA 86: 8407-8411), are a family of polypeptides synthesized by eukaryotic and prokaryotic cells in response to heat shock or other kinds of environmental stresses. The HSP are encoded by a cellular subgroup of genes, identified as stress genes.

The stress genes transcription is regulated by the transcriptional factor HSF (heat shock transcription factor) which is activated in response to temperature increases, environmental stress or after exposition to some biological molecules (Morimoto et al., 1992, J. Biol. Chem. 267: 21987-21990, 1992; Amici et al., 1992, Proc. Natl. Acad Sci USA 89: 6227-6231). The cytoprotective role of stress proteins has been described in various kinds of pathologies, including ischemia, (Marber et al., 1994, J. Cli. Invest. 93: 1087-1094), trauma, inflammation and viral replication (Feige et al., “Stress-Inducible Cellular response” Birkhauser. Verlag, Basel, 1996) to name a few. Heat shock proteins have been shown to interfere at various levels with viral replication, and in particular a cytoprotective role of the HSP70 protein has been characterized in some experimental models of acute infection (M. G. Santoro, Experientia, Vol. 50, 1039-1047, 1994). Therefore, the discovery of compounds that can selectively induce the expression of “heat shock” (hs) proteins in humans would be beneficial for the treatment of a variety of diseases and disorders.

### 2.2 NF-κB

NF-κB (Nuclear Factor-kappaB or Nuclear Factor-κB) is a eukaryotic transcription factor of the rel family, which is normally located in the cytoplasm in an inactive complex, whose predominant form is a heterodimer composed of p50 and p65 subunits, bound to inhibitory proteins of the IκB family (Thanos et al., 1995, Cell 80:529-532).

The inactive form of NF-κB is localized in the cytoplasm, and upon activation by a variety of agents (e.g., cytokines, oxygen free radicals, inhaled particles, ultraviolet light, bacterial products, and viral products) is translocated to the nucleus. NF-κB is tightly associated with a class of specific inhibitory proteins, called IκBs, that prevent the translocation and DNA binding of the transcription factor (see, e.g., Chen et al., 1999, Clinical Chemistry 45:7-17 and Baeuerle, 1998, Cell 95:729-731). In response to a variety of agents, IκB is phosphorylated in its N-terminal domain by a large multikinase complex, polyubiquitinylated, and degraded by the proteasome (see, e.g., Baeuerle, 1998, Curr. Biol. 8:R19-R22; Ghosh et al., 1998, Annu. Rev. Immunol. 16:225-260). Once NF-κB is dissociated from IκB, it translocates to the nucleus and initiates the transcription of genes by binding to its cognate DNA motifs in the regulatory segments of genes. The active form of NF-κB induces the transcription of a variety of genes encoding proteins involved in controlling the immune and inflammatory responses, including genes encoding cytokines (e.g., interleukins and tumor necrosis factor alpha), NO synthase, cyclo-oxygenate-z, chemokines, growth factors, cell adhesion factors and acute phase proteins.

NF-κB is an early mediator of the immune and inflammatory responses, and it is involved in the control of cell proliferation and in the pathogenesis of various human diseases, including, but not limited to, rheumatoid arthritis (Beker et al., 1995, Clin. Exp. Immunol. 99: 325), ischemia (Salminen et al., 1995, Biochem. Biophys. Res. Comm. 212: 939), arteriosclerosis (Baldwin et al., 1996, Annals Rev. Immunol. 14: 649), autoimmune arthritis, asthma, septic shock, lung fibrosis, glumerulonephritis, and acquired immunodeficiency syndrome (AIDS). Many viruses, including human immunodeficiency virus-1 (HIV-1) and human T-cell leukemia virus type I (HTLV-1), utilize NF-κB to their transcriptinal advantage during infection. For example, the transcription of HIV-1 virus RNAs by NF-κB is caused by the presence of KB-sites in the (LTR) (Long Terminal Repeats) sequences of the virus genome (Baltimore et al., 1989, Cell 58: 227-229). Therefore, the discovery of compounds that downregulate or inhibit NF-κB activation after administration to humans would be beneficial for the treatment of diseases and/or disorders associated with inappropriate or aberrant NF-κB activity.

### 3. SUMMARY OF THE INVENTION

It has been discovered that cyclopentenone compounds that demonstrate both cytoprotective inducing properties and NF-κB inhibitory properties, are effective at inhibiting viral replication and/or infection and protecting against cellular damages resulting from inflammatory responses. In particular, the cyclopentenone compounds of the invention inhibit viral replication by activating intracellular defense responses, including the induction of cytoprotective heat shock proteins and regulating the activity of nuclear factor-κB (NF-κB).

The compounds of the present invention include a novel class of both cytoprotective and antiviral drugs that act on different targets essential for the viral life cycle. In accordance with the present invention, the cyclopentenone compounds of the invention encompass compounds with a cyclopentenone ring structure, including synthetic and natural derivatives of cyclopentenone prostaglandins. More importantly, it has been determined that the α,β-unsaturated ketone (“enone”) moiety is important for achieving the desired activity; this moiety is present in cyclopentenone rings. The preferred cyclopentenone compounds of the present invention exhibit both significant inhibition of NF-κB and viral protein expression, and activation of cytoprotective heat shock proteins, thus activating a unique combination of inhibition of viral replication and activation of intracellular defenses to combat viral infection and/or inflammation. It is this dual activity that renders the present invention particularly valuable for disease treatment.

The present invention relates to therapeutic and prophylactic methods and compositions for the treatment and prevention of disorders related to viral infection and/or inflammation based on cyclopentenone compounds such as synthetic and natural derivatives of cyclopentenone prostaglandins, and therapeutically and prophylactically effective preparation containing a cyclopentenone compound such as a cyclopentenone prostaglandin or derivative thereof. The cyclopentenone compounds that may be used in accordance with the present invention can be identified by their ability to inhibit NF-κB activation and to induce activity of cytoprotective heat shock proteins. The present invention relates to a method of inducing cytoprotective responses and inhibiting NF-κB activation in humans, comprising administering one or more cyclopentenone compounds that induce both the activation of heat shock proteins and the inhibition of NF-κB. The compounds which may be administered in accordance with the invention include compounds with cyclopentenone ring structures and alternatively, serine protease inhibitors. The methods of treatment of the present invention may be used to induce cytoprotective responses and NF-κB inhibitory activities for the treatment of viral infection, inflammation, cancer and related disorders in humans. The methods of treatment of the present invention may also be used when suppression of the immune system is desired.

The present invention also relates to methods of treating or preventing diseases and disorders, including viral infections, inflammatory disorders, and cancer in an animal in need thereof, preferably a human in need thereof, comprising identifying a compound that induces one or more heat shock proteins and downregulates or inhibits NF-κB activation and administering the compound to the animal.

In the examples described infra, a number of cyclopentenone compounds, cyclopentenone prostaglandins and derivatives thereof, including cyclopentenone itself and Δ¹²-Prostaglandin J₂, are shown to induce heat shock proteins, e.g., HSP70 and inhibit NF-κB activation in vitro, and to inhibit viral replication, including vesicular stomatitis virus, herpes simplex virus and Sendai virus. The cyclopentenone compounds of the invention are also shown to exhibit a number of antiinflammatory responses, including inhibition of nitrate formation. It is further shown that Δ¹²-PGJ₂, a natural cyclopentenone prostaglandin derivative, is a potent inhibitor of influenza A virus both in vitro and in vivo.

Further, in an alternative embodiment, in the examples described infra, a number of serine protease inhibitors, including 3,4-clair-iso-coumarine (DCIC), tosyl-L-phenylalanine-chloromethylketone (TPCK), Na-tosyl-lysine-chloromethylkenone (TLCK), N-acetyl-DL-phenylalanine-β-ethylester (BTEE), are shown to induce HSP70 (e.g., HSP70), inhibit NF-κB activation, and to inhibit viral replication.

### 4. DESCRIPTION OF THE FIGURES

FIG. 1A-B. Effect of 2-cyclopenten-1-one treatment on HSF activation. Whole cell extracts prepared at different times after treatment with 500 μM 2-cyclopenten-1-one or 3 hours after heat shock were analyzed by EMSA. The positions of heat shock transcription factor-DNA binding complexes (HSF), constitutive HSE binding activity (CHBA) and non-specific protein-DNA interaction (NS) are indicated in FIG. 1A. The levels of HSF-HSE complexes were quantitated by Molecular Dynamics Phospholmager (MDP) analysis and the results are shown in FIG. 1B. HSF values were normalized to the level of HSF DNA binding activity at 9 hours after treatment, which was given a value of 100%.

FIG. 2A-B. Effect of 2-cyclopenten-1-one treatment on heat shock gene transcription. FIG. 2A depicts the autoradiogram results from the transcription run-on assay. The rate of transcription was quantitated by MDP analysis and results are shown FIG. 2B.

FIG. 3A-B. Effect of 2-cyclopenten-1-one treatment on protein synthesis. FIG. 3A depicts the autoradiogram results from the protein synthesis assay. The HSP70 protein synthesis (◯) was determined by densitometric analysis of the autoradiograms and the results are shown in FIG. 3B. Total protein synthesis (●) was determined as [³⁵S]-methionine incorporation into trichloracetic acid-insoluble material.

FIG. 4A-B. Effect of 2-cyclopenten-1-one on VSV replication and protein synthesis. FIG. 4A shows the effect of different concentrations of 2-cyclopenten-1-one on VSV replication as measured by CPE 50%. FIG. 4B shows the effect of 2-cyclopenten-1-one on VSV protein synthesis. Cell lysates of [³⁵S]-methionine labeled uninfected (U) or VSV infected MA104 cells treated with 250 μM of 2-cyclopenten-1-one (lanes 2 and 5), 500 μM 2-cyclopenten-1-one (lanes 3 and 6), or control diluent (lanes 1 and 4) were analyzed by SDS-PAGE and autoradiography. The position of HSP70, identified by western blot analysis using anti-human HSP70 antibodies (data not shown), is indicated by the arrow. VSV proteins L, G, N, WS and H are indicated.

FIG. 5A-B. Dose-dependent activation of HSF and inhibition of NF-κB activation by 2-cyclopenten-1-one. Whole cell extracts prepared 3 hours after treatment with different concentrations (125-500 μM) of 2-cyclopenten-1-one and 25 ng/ml TPA (12-o-tetradecanoyl-phorbol-13-acetate) were analyzed by EMSA. The positions of NF-κB-DNA complexes (NF-κB) and non-specific protein-DNA interaction (NS) are indicated in FIG. 5A. The positions of heat shock transcription factor-DNA binding complexes (HSF), constitutive HSE binding activity (CHBA) and non-specific protein-DNA interaction (NS) are indicated in FIG. 5B.

FIG. 6A-B. Specificity of the chemical structure which is responsible for NF-κB inhibition and HSF activation. Whole cell extracts prepared 3 hours after treatment with 500 μM of 2-cyclopenten-1-one, 500 μM of cyclopentanone, or 500 μM of cyclopentene and 25 ng/ml TPA (12-o-tetradecanoyl-phorbol-13-acetate) were analyzed by EMSA. The positions of NF-κB-DNA complexes (NF-κB) and non-specific protein-DNA interaction (NS) are indicated in FIG. 6A. The positions of heat shock transcription factor-DNA binding complexes (HSF), constitutive HSE binding activity (CHBA) and non-specific protein-DNA interaction (NS) are indicated in FIG. 6B.

FIG. 7. Effect of DCIC on HSF activation. Whole cell extracts prepared at different times after treatment with 5 μM DCIC were analyzed by EMSA. The positions of heat shock transcription factor-DNA binding complexes (HSF), constitutive HSE binding activity (CHBA) and non-specific protein-DNA interaction (NS) are indicated in FIG. 7A. The levels of HSF-HSE complexes were quantitated by Molecular Dynamics Phospholmager (MDP) analysis and the results are shown in FIG. 7B. HSF values were normalized to the level of HSF DNA binding activity at 3 hours after treatment, which was given a value of 100%.

FIG. 8. Effect of DCIC treatment on heat shock gene transcription. FIG. 8A depicts the autoradiogram results from the transcription run-on assay. The rate of transcription was quantitated by MDP analysis and results are shown FIG. 8B.

FIG. 9. Effect of DCIC on VSV replication and protein synthesis. FIG. 9A shows the effect of different concentrations of DCIC on VSV replication as measured by CPE 50%. FIG. 9B shows the effect of DCIC on VSV protein synthesis. Cell lysates of [³⁵S]-methionine labeled uninfected (U) or VSV infected MA104 cells treated with 5 μM of DCIC (lanes 2 and 7), 15 μM of DCIC (lanes 3 and 8), 30 μM of DCIC (lanes 4 and 9), 45 μM of DCIC (lanes 5 and 10), or control diluent (lanes 1 and 6) were analyzed by SDS-PAGE and autoradiography. The position of HSP70, identified by western blot analysis using anti-human HSP70 antibodies (data not shown), is indicated by the arrow. VSV proteins L, G, N, WS and H are indicated. shows the antiviral activity of DCIC. FIG. 9B shows the induction of the HSP70 and the inhibition of the synthesis of the viral proteins by DCIC.

FIG. 10. Dose-dependent activation of HSF and inhibition of NF-κB activation by serine protease inhibitors. Whole cell extracts prepared 3 hours after treatment with different concentrations of DCIC, PLCK or TPCK and 25 ng/ml TPA (12-o-tetradecanoyl-phorbol-13-acetate) were analyzed by EMSA. The positions of NF-κB-DNA complexes (NF-κB), non-specific protein-DNA interaction (NS), heat shock transcription factor-DNA binding complexes (HSF), constitutive HSE binding activity (CHBA) and non-specific protein-DNA interaction (NS) are indicated herein. The line “control” refers to cells non-stimulated with TPA as reference of non-activated NF-κB.

FIG. 11. Effect of Δ¹²-PGJ₂ on PR8 influenza virus replication and protein synthesis. A) MDCK cells infected with PR8 influenza virus were treated with different concentrations of Δ¹²-PGJ₂ soon after the 1 hour adsorption period. Virus yield was determined 24 h p.i. by HAU titration. B) Under the same conditions, PR8 virus replication was inhibited by more than 95% up to 72 h p.i. after a single treatment with 6 μg/ml A¹² PGJ₂ ( ) as compared to control (◯). Data in A and B represent the mean±SD of at least duplicate samples of a representative experiment. Each experiment was repeated at least 3 times. The Student's t test was used for unpaired data analysis and P values <0.05 were considered significant. *=P<0.05. C-E, MDCK cells mock-infected (U) or infected with PR8 influenza virus (PR8) were treated with Δ¹²-PGJ₂ (6 μg/ml) (+) or control diluent (−) soon after the 1 h adsorption period, and labeled with [³⁵S]methionine for the following 24 hours. Protein synthesis in uninfected (□) or PR8-infected ( [symbol] ) cells, as determined by [³⁵S]methionine incorporation into trichloroacetic acid-insoluble material, is shown in panel E. Samples containing equal amounts of radioactivity were processed for SDS-PAGE and autoradiography (C). Samples containing equal amounts of protein were processed for 1B analysis using anti-hsp70 antibodies which recognize both the 72-kDa constitutive hsc70 and the 70-kDa inducible hsp70 (D). Viral proteins HA, NP and M1 are indicated.

FIG. 12. Effect of different prostaglandins on PR8 virus replication. MDCK cells infected with PR8 influenza virus (1 HAU/ml) were treated with 6 μg/ml of prostaglandin A₁ (PGA₁), D₂ (PGD₂), E₂ (PGE₂), J₂ (PGJ₂), Δ¹²-PGJ₂ or ethanol diluent soon after the 1 h adsorption period. Virus yield was determined at 24 hours (panel A) and 48 hours (panel B) p.i. by HAU titration. Data represent the mean±SD of triplicate samples. *=P<0.05.

FIG. 13. Effect of Δ¹²-PGJ₂ on DNA and RNA synthesis in uninfected or PR8-infected MDCK cells. Confluent MDCK monolayers mock-infected (U) or infected with PR8 influenza virus (PR8) were treated with Δ¹²-PGJ₂ (6 μg/ml) ( [symbol] ) or control diluent (□) soon after the 1 h adsorption period, and labeled with [³H]uridine (A,B) or [³H]thymidine (C,D). The amount of radioactivity incorporated into the TCA-soluble (uptake; A, C) or -insoluble (incorporation; B, D) material was determined after 24 hours. Data represent the mean±SD of at least duplicate samples of a representative experiment.

FIG. 14. Effect of Δ¹²-PGJ₂ on the kinetics of PR8 virus protein synthesis. MDCK cells mock-infected (U) or infected with PR8 influenza virus (1 HAU/ml)(PR8) were treated with Δ¹²-PGJ₂ (6 μg/ml) (+) or control diluent (−) soon after the 1 hour adsorption period, and labeled with [³⁵S]methionine for 45 minutes at the times indicated. A.) Samples containing equal amounts of radioactivity were processed for SDS-PAGE and autoradiography. HSP70 is indicated by the arrow. Asterisk indicates a 32 kDa protein, which was identified as heme-oxygenase by immunoblot (IB) analysis (data not shown). B) Samples containing equal amounts of protein were processed for IB analysis using a polyclonal anti-WSN virus antiserum which recognizes PR8 virus HA, NP and M1 proteins (1).

FIG. 15. Effect of Δ¹²-PGJ₂-treatment on the survival of PR8 influenza virus-infected in ice. Balb/c mice were infected i.n. with 100 μl/mouse of PR8 virus suspension (12.5 HAU/ml) on day 0, and treated i.p. with different doses of Δ¹²-PGJ₂. A) (◯) control diluent (n=10); (▴) Δ¹²-PGJ₂ (1 μg/day/mouse on days 1 to 7 p.i.; n=10); (▪) Δ¹²-PGJ₂ (5 μg/day/mouse on days 1 to 7 P.I.; n=10). B) (◯) control diluent (n=20); (A) Δ¹²-PGJ₂ (5 ng/day/mouse on day 0, 2 and 4 p.i.; n=20); (▪) Δ¹²-PGJ₂ (5 μg/day/mouse on days 1 to 7 p.i.; n=20). Percent survival was significantly increased in mice treated with Δ¹²-PGJ₂ (5 μg/day/mouse on days 1 to 7) as compared to control both in A and B (α<0.005).

### 5. DETAILED DESCRIPTION OF THE INVENTION

The present invention relates to cyclopentenone compounds, including synthetic and natural derivatives of cyclopentenone prostaglandins which have potent NF-κB inhibitory activities and cytoprotective inducing activities. As a result of their unique combination of activities, the cyclopentenone compounds of the present invention are effective at inhibiting viral replication and/or infection, protecting against cellular damages resulting from inflammatory responses in humans and useful in treating cancer and numerous other diseases. The present invention also relates to cyclopentenone prostaglandins and derivatives thereof which decrease viral load and/or treat or prevent disorders associated with viral infection. The cyclopentenone compounds of the present invention inhibit NF-κB activation, preferably by preventing degradation or phosphorylation of the NF-κB inhibitor or by activation of serine proteases which inhibit activation of NF-κB. The cyclopentenone compounds of the present invention also induce cytoprotective responses, preferably through induction of heat shock protein, including inducing transcription, expression, activation, translocation and phosphorylation of heat shock proteins. Thus, the cyclopentenone compounds, prostaglandins and derivatives thereof which may be used in accordance with the present invention can be identified by their ability to both inhibit NF-κB and induce cytoprotective responses.

The present invention relates to a method of inducing cytoprotective responses and inhibiting NF-κB activation in humans, comprising administering an effective amount of one or more cyclopentenone compounds that induce both the activation of heat shock proteins and the inhibition of NF-κB. The compounds which may be administered in accordance with the invention include compounds with a cyclopentenone ring structure and alternatively, serine protease inhibitors. The methods of treatment of the present invention may be used to induce cytoprotective responses and NF-κB inhibitory activities for the treatment of viral infection, inflammation, cancer and related disorders in humans. Further, in accordance with the present invention, the cyclopentenone compounds or prostaglandins may be administered to humans when suppression of the immune system is desired, e.g., treatment of auto-immune disorders or to facilitate acceptance of transplants.

The present invention relates to pharmaceutical compositions containing cyclopentenone compounds, prostaglandins and derivatives thereof and methods of administering those compositions for the treatment and/or prevention of viral infection and/or replication in humans. The cyclopentenone compounds of the present invention are potential antiviral agents, in that they act on different targets essential for the viral life cycle and simultaneously activate intracellular defenses to combat viral infection. In particular, the compounds of the present invention act to inhibit viral mRNA transcription and translation and block viral protein synthesis. Given the diverse range of targets of the cyclopentenone compounds of the invention, they have utility in combating viral infection prophylactically, i.e., to prevent viral infection, or they can be administered at late stages of the viral replication life cycle and still effectively reduce viral load and inhibit viral replication. The cyclopentenone compounds of the present invention are particularly useful in inhibiting replication of RNA viruses, including Sendai virus, Influenza virus, respiratory syncitial virus, polio virus, vesicular stomatitis virus and human immunodeficiency virus, and replication of DNA viruses, including pox viruses and herpes viruses.

Efficacy in treating or preventing viral infection may be demonstrated by detecting the ability of the cyclopentenone compound or derivative thereof to inhibit the replication of the virus, to inhibit transmission or prevent the virus from establishing itself in its host, or to prevent, ameliorate or alleviate the symptoms of disease a progression. The treatment is considered therapeutic if there is, for example, a reduction is viral load, amelioration of one or more symptoms, or a decrease in mortality and/or morbidity following administration of the compounds of the invention.

The present invention relates to pharmaceutical compositions suitable for administration to humans containing cyclopentenone compounds such as prostaglandins and derivatives thereof and methods of administering these compositions for the treatment and prevention of a number of disorders, including inflammation and the cell damage resulting from inflammation. The cyclopentenone compounds of the present invention induce cytoprotective responses, including the activation of heat shock proteins. The compounds of the present invention have many beneficial effects, including the induction of heat proteins, in particular HSP70, which induces a thermotolerant state in cells, thus preventing lethal injury and preserving cellular function and homeostasis. The cyclopentenone compounds of the present invention are particularly useful in treating or preventing inflammation, ischemia, trauma and hyperthernia. Efficacy in treating or preventing these disorders may be demonstrated by the amelioration or alleviation of any of symptoms associated with disease progression.

In preferred embodiments, the invention provides a therapeutic composition comprising a cyclopentenone compound selected from the following, Δ¹²-Prostaglandin J₂, 2-cyclopenten-1-one, or any combination thereof, and methods of administering these compositions for the treatment and prevention of infections and inflammatory disorders. In another embodiment, the invention provides a therapeutic composition comprising a serine protease inhibitor selected from the following, 3,4-clair-iso-coumarine (DCIC), tosyl-L-phenylalanine-chloromethylketone (TPCK), Na-tosyl-lysine-chloromethylkenone (TLCK), -acetyl-DL-phenylalanine-β-ethylester (BTEE), or any combination thereof, and methods of administering these compositions for the treatment and prevention of infections and inflammatory disorders.

### 5.1. Compounds of the Invention

It has been discovered that compounds with an α,β-unsaturated ketone (“enone”) moiety are the preferred compounds of the invention. The enone moiety may be present in a ring or in an acyclic structure, for example, cyclopentenone, cyclohexenone, cycloheptone and the like or simple acyclic α,β-unsaturated carbon chains may be used. The preferred most compounds of the present invention comprise compounds with a cyclopentenone ring structure. The cyclopentenone containing compounds may or may not contain long aliphatic lateral side chains similar to those present in prostaglandins or punaglandins that have a cyclopentenone ring structure (sometimes referred to as a cyclopentenone nucleus). Accordingly, the compounds may lack one or more long aliphatic lateral side chains at the 4 and/or 5 positions of the cyclopentenone ring.

In one embodiment, cyclopentenone containing compounds of the invention include those compounds which have cytoprotective including activities, including activation of one or more heat shock proteins, preferably HSP70. In another embodiment, cyclopentenone containing compounds of the invention include those compounds that have NF-κB inhibitory activities. In a preferred embodiment, cyclopentenone containing compounds of the invention include those compounds that exhibit a combination of cytoprotective inducing activities and NF-κB inhibitory activities, and are effective at inhibiting viral replication and protecting against cellular damages resulting from inflammatory responses in humans.

The cyclopentenone containing compounds of the invention include, but are not limited to: prostaglandins, analogs and derivatives thereof; 2-cyclopenten-1-one; and derivatives of 2-cyclopenten-1-one.

In a preferred embodiment, cyclopentenone compounds of the invention have equal or higher activity than cyclopent-2-en-1-one in respect to one or more the following: activating HSF, inhibiting NF-κB, and inhibiting viral replication (i.e., inhibiting the viral replication of HSV-1 or Sendai virus). In accordance with this embodiment, the equal or increased activity of the compound relative to cyclopent-2-en-1-one need not exist at all concentrations. However, it is preferred that the activity of the compound relative to cyclopent-2-en-1-one exist over a range of 1-10 μM, 1-25 μM, 1-50 μM, 1-75 μM, 1-100 μM, 1-125 μM, 1-150 μM, 1-175 μM, or 1-200 μM. Preferably, the compound has a level of activity at least 1.5 times, at least 2 times, at least 3 times, at least 4 times, at least 5 times, at least 6 times, at least 7 times, at least 8 times, at least 9 times, at least 10 times, at least 15 times, at least 25 times, at least 50 times, at least 75 times or at least 100 times the level of activity of cyclopent-2-en-1-one.

Prostaglandin compounds of the invention include, but are not limited to, prostaglandins of the A type and metabolites and analogs thereof (e.g., PGA₁, PGA₂ and 16,16-dimethyl-PGA₂); prostaglandins of the J type and metabolites and analogs thereof (e.g., PGJ₂ and 15-deoxy Δ¹²⁻¹⁴-PGJ₂); and prostaglandins of the D type and metabolites and analogs thereof (e.g., PGD₂ and 9-deoxy-Δ⁹,Δ¹²-13,14-dihydro-PGD₂ (Δ¹²-PGJ₂)).

In an alternative embodiment, the compounds of the invention also include serine protease inhibitors that induce one or more heat shock proteins, preferably HSP70. The compounds of the invention also include serine protease inhibitors that downregulate or inhibit NF-κB activity. Further, the compounds of the invention include serine protease inhibitors that induce one or more heat shock proteins and -downregulate or inhibit NF-κB activity. Examples of such serine protease inhibitors include, but are not limited to, 3,4-dichloro-iso-coumarine (DCIC), tosyl-L-phenylalanine-chloromethylketone (TPCK), N_{α}-tosyl-lysine-chloromethylketone (TLCK), N-acetyl-DL-phenylalanine-α-napthylester (APNE), and N-benzoyl-L-thyroxine-ethylester (BTEE).

It should be appreciated that certain compounds of the invention may contain one or more chiral atoms. Thus, the invention encompasses all stereoisomers, including enatiomers, diastereoisomers and mixtures thereof. In a preferred embodiment, the invention includes the racemic or either the R- or S-enantiomers of all the compounds described herein. The enantiomers may each be provided in a form substantially free of the other enantiomer (e.g., at least 75% free (w/w), at least 90% free (w/w) or at least 99% free (w/w)) or as mixtures (e.g., racemic mixtures). The compounds of the invention can be isolated from natural sources using standard purification techniques such as, for example, chromatography (e.g., ion exchange, affinity, particularly by affinity for the specific antigen after Protein A, and sizing column chromatography), centrifugation, and differential solubility, or can be chemically synthesized.

### 5.2. Uses of Compounds of the Invention

The present invention encompasses therapeutic methods and compositions for the treatment, prevention or inhibition of diseases and disorders, including infectious diseases (e.g., microbial and viral infections), immune disorders, cancer, ischemia and arteriosclerosis, comprising one or more compounds with a cyclopentenone ring structure. In one embodiment, therapeutic methods and pharmaceutical compositions for treating, inhibiting or preventing infectious diseases, immune disorders, cancer, ischemia and arteriosclerosis in animals, including humans, comprise one or more prostaglandins or prostaglandin derivatives containing an α,β-unsaturated carbonyl group in a cyclopentane ring (a cyclopentenone ring structure), with the proviso that the prostaglandin is not PGD₂, Δ-13, 14-dihydro-PGD₂ (Δ¹²-PGJ₂), PGA₂, 15-deoxy-13,14-dihydroprostaglandin J₂, racemic 4-tert-butyldimethylsilyloxy-cyclopent-2-en-1-one or the compound depicted below.

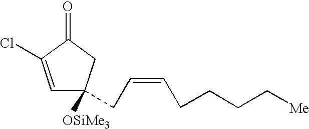

In a specific embodiment, the following known prostaglandins are used in therapeutic methods and compositions in accordance with the present invention for treating, inhibiting or preventing infectious diseases, immune disorders, cancer, ischemia and arteriosclerosis in animals, including humans: PGJ₂, 15-deoxy Δ¹²⁻¹⁴-PGJ₂, and PGA₁. In a preferred embodiment, the following known prostaglandins are used in therapeutic methods and compositions in accordance with the present invention for treating, inhibiting or preventing infectious diseases, immune disorders, cancer, ischemia and arteriosclerosis in humans: PGA₁, PGA₂,PGA₂ 16,16-dimethyl-PGA₂, PGD₂, 9-deoxy-Δ⁹,Δ¹²-13,14-dihydro-PGD₂ (Δ¹²-PGJ₂), PGJ₂, 15-deoxy-13,14-dihydroprostaglandin J₂ and 15-deoxy Δ¹²⁻¹⁴-PGJ₂.

In another embodiment, therapeutic methods and pharmaceutical compositions for treating, inhibiting or preventing infectious diseases, immune disorders, cancer, ischemia and arteriosclerosis in animals, comprise one or more serine protease inhibitors that induce one or more heat shock proteins, preferably HSP70, and downregulate or inhibit NF-κB activity. Examples of such serine protease inhibitors include, but are not limited to, 3,4-dichloro-iso-coumarine (DCIC), tosyl-L-phenylalanine-chloromethylketone (TPCK), N_{α}-tosyl-lysine-chloromethylketone (TLCK), N-acetyl-DL-phenylalanine-p-napthylester (APNE), and N-benzoyl-L-thyroxine-ethylester (BTEE). In yet another embodiment, therapeutic methods and pharmaceutical compositions for treating, inhibiting or preventing infectious diseases, immune disorders, cancer, ischemia and arteriosclerosis in animals, comprise one or more cyclopentenone compounds of the invention in combination with one or more serine protease inhibitors of the invention.

In a preferred embodiment, therapeutic methods and pharmaceutical compositions for treating, inhibiting or preventing infectious diseases, immune disorders, cancer, ischemia, arteriosclerosis and diabetes in animals, comprise 2-cyclopenten-1-one or a derivative of 2-cyclopenten-1-one.

In a preferred embodiment, therapeutic methods and pharmaceutical compositions for treating, inhibiting or preventing infectious diseases, immune disorders, cancer, ischemia, arteriosclerosis and diabetes in animals, comprise cyclopentenone containing compounds of the invention having equal or higher activity than cyclopent-2-en-1-one in respect to one or more the following: activating HSF, inhibiting NF-κB, and inhibiting viral replication (i.e., inhibiting the viral replication of HSV-1 or Sendai virus).

In a preferred embodiment, therapeutic or pharmaceutical compositions are administered to an animal to treat, prevent or inhibit infectious diseases. Infectious diseases include diseases associated with yeast, fungal, viral and bacterial infections. Viruses causing viral infections which can be treated or prevented in accordance with this invention include, but are limited to, retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus, HHV6-HHV8, and cytomegalovirus), arenavirues (e.g., lassa fever virus), paramyxoviruses (e.g., morbillivirus virus, human respiratory syncytial virus, mumps, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C), papovaviruses (e.g., papillomavirues), picornaviruses (e.g., rhinoviruses, enteroviruses and hepatitis A viruses), poxviruses, reoviruses (e.g., rotavirues), togaviruses (e.g., rubella virus), rhabdoviruses (e.g., rabies virus). Microbial pathogens causing bacterial infections include, but are not limited to, Streptococcus pyogenes, Streptococcus pneumoniae, Neisseria gonorrhoea, Neisseria nieningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori.

In another embodiment, therapeutic or pharmaceutical compositions are administered to an animal to treat, prevent or inhibit immune disorders. Immune disorders include, but are not limited to, autoimmune disorders (e.g., arthritis, graft rejection, Hashimoto's thyroiditis, and insulin-dependent diabetes), inflammatory disorders (e.g., bacterial infection, psoriasis, septicemia, cerebral malaria, inflammatory bowel disease, arthritis, gastroenteritis, and glomerular nephritis), and allergic inflammatory disorders (e.g., asthma, allergic rhinitis, and contact dermatitis).

In another embodiment, therapeutic or pharmaceutical compositions are administered to an animal to treat, prevent or inhibit cancer and proliferative disorders. Examples of types of cancer, include, but are not limited to, leukemia (e.g., acute leukemia such as acute lymphocytic leukemia and acute myelocytic leukemia), neoplasms, tumors (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma), heavy chain disease, metastases, or any disease or disorder characterized by uncontrolled cell growth.

In another embodiment, therapeutic or pharmaceutical compositions are administered to an animal to treat, prevent or inhibit disorders mediated by radiation. Examples of such disorders include, but are not limited to, cell and tissue trauma, and cell and tissue aging. In another embodiment, therapeutic or pharmaceutical compositions are administered to an animal to treat, prevent or inhibit ischemia and arteriosclerosis. Examples of such disorders include, but are not limited to, reperfusion damage (e.g., in the heart and/or brain) and cardiac hypertrophy. In another embodiment, therapeutic or pharmaceutical compositions are administered to an animal to treat, prevent or inhibit disorders involving damage to or killing of cells. Examples of such disorders include, but are not limited to, disorders resulting from chemical toxicity, oxidative cell damage, cell and tissue aging, trauma, and diabetes. In yet another embodiment, therapeutic or pharmaceutical compositions are administered to an animal to treat, prevent or inhibit neurological disorders. Examples of such disorders include, but are not limited to, neuropsychiatric disorders (e.g., schizophrenia, attention deficient disorders, shizoaffective disorders, bipolar affective disorders and unipolar affective disorders), neuromuscular disorders (e.g., progressive spinal muscular atrophy), neurodegenerative disorders (e.g., Alzheimer's disease, stroke, dementia, Parkinson's disease, and Huntington's disease), demyelinating diseases (e.g., multiple sclerosis, multiple pontine myelinolysis, human immunodeficiency associated myelopathy, and transverse myelopathy), spinal cord injuries, malignant lesions (e.g., glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma), brain injuries, and infectious lesions.

In a specific embodiment, an animal is administered a compound of the present invention in an amount effective for the treatment, prevention or inhibition of a disease or disorder such as an infectious disease, or an amount effective such that the viral titers decrease, or an amount effective such that bacterial counts decrease. In another specific embodiment, an animal is administered a compound of the present invention in an amount effective for inducing an anti-tumor response (e.g., the inhibition of the hyperproliferation of a tumor), or an amount effective such that the immune response in an animal is modified (i.e., increased or decreased). In a preferred embodiment, an animal is administered a compound of the present invention in an amount effective to induce heat shock factor (HSF) or one or more heat shock proteins, preferably HSP70. In another preferred embodiment, an animal is administered a compound of the present invention in an amount effective to downregulate or inhibit NF-κB activity. In a most preferred embodiment, an animal is administered a compound of the present invention in an amount effective to induce heat shock factor (HSF) or one or more heat shock proteins, preferably HSP70, and to downregulate or inhibit NF-κB activity.

One or more compounds of the invention can be administered to an animal for the treatment or prevention of diseases or disorders such as cancer, immune disorders, neurological disorders or infectious diseases in combination with one or more known compounds used to treat or prevent such diseases or disorders. In one embodiment, an animal is administered one or more cyclopentenone compounds of the invention in combination with one or more known antiviral agents for the treatment, prevention or inhibition of a viral infection. In another embodiment, an animal is administered one or more serine protease inhibitors of the invention in combination with one or more known antiviral agents for the treatment, prevention or inhibition of a viral infection. Examples of antiviral agents include, but are not limited to, acyclovir, AZT, interferon, and amantadine. In another embodiment, an animal is administered one or more cyclopentenone compounds of the invention in combination with one or more known anti-inflammatory agents (e.g., aspirin) for the treatment, prevention or inhibition of immune disorders (e.g., inflammatory disorders).

### 5.3. Demonstration of Therapeutic or Prophylactic Uses of Compounds

The compounds of the invention are preferably tested in vitro, and then in vivo for the desired therapeutic or prophylactic activity, prior to use in humans. For example, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed. In various specific embodiments, in vitro assays can be carried out with representative cells of cell types involved in a patient's disorder, to determine if a compound has a desired effect upon such cell types. Preferably, the compounds of the invention are also tested in in vitro assays and animal model systems for their toxicity prior to administration to humans.

Compounds for use in therapy can be tested for their toxicity in suitable animal model systems, including but not limited to rats, mice, chicken, cows, monkeys, and rabbits. For in vivo testing of a compound's toxicity any animal model system known in the art may be used.

Efficacy in treating or preventing viral infection may be demonstrated by detecting the ability of the cyclopentenone compound to inhibit the replication of the virus, to inhibit transmission or prevent the virus from establishing itself in its host, or to prevent, ameliorate or alleviate the symptoms of disease a progression. The treatment is considered therapeutic if there is, for example, a reduction is viral load, amelioration of one or more symptoms or a decrease in mortality and/or morbidity following administration of a compound of the invention.

Compounds of the invention can be tested for their ability to modulate HSF activity and/or NF-κB activity by contacting cells, preferably human cells, with a compound of the invention or a control compound and determining the ability of the compound of the invention to modulate HSF activity and/or NF-κB activity. Techniques known to those of skill in the art can be used to measure a compound's ability to modulate the HSF and NF-κB activation (see, e.g., the Example Section infra). For example, HSF and NF-κB activation can be measured by electrophoretic shift assays and reporter assays.

Compounds of the invention can be tested for the ability to induce the expression of heat shock proteins (e.g., HSP70 expression), by contacting cells, preferably human cells, with a compound of the invention or a control compound and determining the ability of the compound to induce one or more heat shock proteins. Techniques known to those of skill in the art can be used to measure the level of expression of heat shock proteins (see, e.g., the Example Section infra). For example, the level of expression of heat shock proteins can be measured by analyzing the level of RNA of heat shock proteins by, for example, RT-PCR and Northern blot analysis, and by analyzing the level of heat shock proteins by, for example, immunoprecipitation followed by western blot analysis and ELISA.

Compounds of the invention can be tested for their ability to modulate the biological activity of immune cells by contacting immune cells, preferably human immune cells (e.g., T-cells, B-cells, and Natural Killer cells), with a compound of the invention or a control compound and determining the ability of the compound of the invention to modulate (i.e, increase or decrease) the biological activity of immune cells. The ability of a compound the invention to modulate the biological activity of immune cells can be assessed by detecting the expression of cytokines or antigens, detecting the proliferation of immune cells, detecting the activation of signaling molecules, detecting the effector function of immune cells, or detecting the differentiation of immune cells. Techniques known to those of skill in the art can be used for measuring these activities. For example, cellular proliferation can be assayed by ³H-thymidine incorporation assays and trypan blue cell counts. Cytokine and antigen expression can be assayed, for example, by immunoassays including, but are not limited to, competitive and non-competitive assay systems using techniques such as western blots, immunohisto-chemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electrophoretic shift assays (EMSAs). The effector function of T-cells can be measured, for example, by a ⁵¹Cr-release assay (see, e.g., Palladino et al., 1987, Cancer Res. 47:5074-5079 and Blachere et al., 1993, J. Immunotherapy 14:352-356).

Compounds of the invention can be tested for their ability to reduce tumor formation in in vitro, ex vivo and in vivo assays. Compounds of the invention can also be tested for their ability to inhibit viral replication or reduce viral load in in vitro and in vivo assays (see the Example section infra). Compounds of the invention can also be tested for their ability to reduce bacterial numbers in in vitro and in vivo assays known to those of skill in the art. Compounds of the invention can also be tested for their ability to alleviate of one or more symptoms associated with cancer, an immune disorder (e.g., an inflammatory disease), a neurological disorder or an infectious disease. Compounds of the invention can also be tested for their ability to decrease the time course of the infectious disease. Further, compounds of the invention can be tested for their ability to increase the survival period of animals suffering from disease or disorder, including cancer, an immune disorder or an infectious disease. Techniques known to those of skill in the art can be used to analyze the function of the compounds of the invention in vivo.

### 5.4. Therapeutic and Pharmaceutical Compositions and Administration Thereof

The invention provides methods of treatment (and prophylaxis) by administration to an animal of an effective amount of a compound of the invention. In a preferred aspect, the compound is substantially purified (e.g., substantially free from substances that limit its effect or produce undesired side-effects). The term “animal” as used herein includes, but is not limited to, animals such as cows, pigs, horses, chickens, cats, dogs, etc., and is preferably a mammal, and most preferably human. In a specific embodiment, a non-human mammal is the animal being treated by administrating of a compound of the invention.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu & Wu, 1987, J. Biol. Chem. 262:4429-4432), construction of a nucleic acid as part of a retroviral or other vector, etc. Methods of introduction include but are not limited to intratumoral, intradermal, intramuscular, intraperitoneal, intravenous, subcutaneous, intranasal, epidural, vaginal, topical, rectal and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

In another embodiment, the compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); WO 91/04014; U.S. Pat. No. 4,704,355; Lopez-Berestein, ibid., pp. 317-327; see generally ibid.)

In yet another embodiment, the compound can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient can be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention are administered via the nasal cavity to the lungs.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilised) condition requiring only the addition of a sterile liquid carrier, e.g., sterile water for injections, immediately prior to use. Extemporaneous injection solutions and suspensions may be prepared from sterile powders, granules and tablets.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the compound of the invention which will be effective in the treatment of viral infection can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, without being bound by any particular dosage, a daily dosage of 10 μg to 100 mg per kilogram of body weight or a daily dosage of 5 μg to 50 mg per kilogram may be suitable for administration to an animal. Specifically, suitable dosage ranges for intravenous administration are generally about 10 to 500 μg of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.5. Screening Assays

The invention provides methods for identifying agents, candidate compounds or test compounds that induce one or more heat shock proteins and/or downregulate or inhibit NF-κB activity. Examples of agents, candidate compounds or test compounds include, but are not limited to, nucleic acids (e.g., DNA and RNA), carbohydrates, lipids, proteins, peptides, peptidomimetics, small molecules and other drugs. Preferably, agents, candidate compounds or test compounds are compounds with a cyclopentenone structure or compounds with an α,β-unsaturated ketone (“enone”) moiety. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and

Felici, 1991, J. Mol. Biol. 222:301-310), each of which is incorporated herein in its entirety by reference.

In one embodiment, agents that induce one or more heat shock proteins and/or downregulate or inhibit NF-κB activity are identified in a cell-based assay system. In accordance with this embodiment, cells are contacted with a candidate compound (e.g., 2-cyclopenten-1-one) or a control compound (e.g., phosphate buffered saline (PBS)) and the ability of the candidate compound to induce one or more heat shock proteins and/or downregulate or inhibit NF-κB activity is determined. The level of expression of one or more heat shock proteins or the downregulation of NF-κB activity in the presence of the candidate compound is compared to the level of expression of one or more heat shock proteins or the downregulation of NF-κB activity in the absence of the candidate compound (e.g., in the presence of a control compound). The candidate compound can then be identified based on this comparison. For example, when the expression of one or more heat shock proteins is significantly greater in the presence of the candidate compound than in its absence, the candidate compound is identified as an inducer of one or more heat shock proteins. The cell, for example, can be of mammalian or human. The ability of the candidate compound to induce one or more heat shock proteins and/or downregulate or inhibit NF-κB activity can be determined by methods known to those of skill in the art. For example, the ability of a candidate compound to induce one or more heat shock proteins can be determined at the RNA level by Northern blot analysis or RT-PCR and at the protein level by immunoprecipitation or western blot analysis. The ability of a candidate compound to downregulate or inhibit NF-κB activity can be determined, for example, by electrophoretic shift assays, by detecting the expression of a protein known to be regulated by NF-κB, detecting the induction of a reporter gene (e.g., an NF-κB regulatory element operably linked to a nucleic acid encoding a detectable marker, e.g., luciferase, β-galactosidase or chloramphenicol (CAT)), or detecting a cellular response, for example, cellular differentiation, or cell proliferation.

In another embodiment, agents that induce one or more heat shock proteins and/or downregulate or inhibit NF-κB activity are identified in an animal model. Examples of suitable animals include, but are not limited to, mice, rats, rabbits, monkeys, guinea pigs, dogs and cats. In accordance with this embodiment, the test compound or a control compound is administered (e.g., orally, rectally or parenterally such as intraperitoneally or intravenously) to a suitable animal and the effect on the expression of one or more heat shock proteins, the inhibition of NF-κB activity, or both expression is determined.

Agents identified that induce more heat shock proteins and/or downregulate or inhibit NF-κB activity can further be tested for there ability to inhibit viral replication in in vitro or in vivo assays. Techniques known to those of skill in the art can be used to measure the inhibition of viral replication (see, e.g., the Example Sections below).

### 6. EXAMPLE

### 2-Cyclopenten-1-One Induces HSP70 Expression, Inhibits Viral Replication, and Inhibits NF-κB Activation

The following example demonstrates the ability of 2-cyclopenten-1-one to induce HSP70 transcription, to inhibit viral replication, and to inhibit NF-κB activation in vitro.

### Materials & Methods

Cell Culture

K562 human erythroleukemia cells, monolayers of monkey kidney cells (MA104 cells), and Jurkat cells were grown in RPMI 1640 medium supplemented with 10% fetal calf serum and antibiotics at 37° C. in a 5% CO₂ humidified atmosphere (Amici et al., 1995, Cancer Research 55: 14452-4457).

Electrophoretic Mobility Shift Assay

K562 cells were treated with 500 μM of 2-cyclopenten-1-one dissolved in ethanol at 37° C. or control diluent (negative control), or stressed at 45° C. for 20 minutes (heat shock treatment; positive control). At different times following 2-cyclopenten-1-one treatment or 3 hours following heat shock, whole cell extracts were prepared and analyzed by EMSA (Electrophoretic Mobility Shift Assay) as described in Amici et al., 1995, supra.

Jurkat cells were treated with different concentrations (125-100 μM) of 2-cyclopenten-1-one for 1 hour and then were stimulated with TPA (25 ng/ml). Alternatively, Jurkat cells were treated for 1 hour with 500 μM cyclopenten-1-one, 500 μM cyclopentanone or 500 μM cyclopentene, and then stimulated with TPA (25 ng/ml). Three hours after treatment, whole cell extracts were prepared and analyzed by EMSA (Electrophoretic Mobility Shift Assay) as described in Amici et al., 1995, supra. After a 20 minute incubation at room temperature, HSF-HSE or NF-κB-DNA-complexes were analyzed by nondenaturing 4% polyacrylamide gel electrophoresis and autoradiography. The amount of shifted HSE probe and NF-κB probe, an indicator of HSF DNA-binding activity and NF-κB activity, respectively, were quantitated by Molecular Dynamics Phospholmager (MDP) analysis.

Transcription Run-On Assay

In vitro run-on transcription reactions were performed in isolated K562 nuclei as described in Banerji et al, 1984, Mol. Cell. Biol. 5: 2437-2448. ³²P-labeled RNA was hybridized to nitrocellulose filters containing plasmids for the following human genes: hsp7O (pH 2.3; Wu et al., 1985, Mol. Cell. Biol. 5: 330-341); grp78/BiP (glucose-regulated 78 protein) (pHG 23.1; Amici et al., 1992, Proc. Natl. Acad. Sci. USA 89, 6227-6231); hsc7O (heat shock cognate70) (pHA 7.6; Amici et al., 1992, Proc. Natl. Acad. Sci. USA 89, 6227-6231); HO (heme oxygenase) (HO clone 2/10; Rossi et al., 1995, Biochem. J. 308: 455-463); and GAPDH (rat glyceraldehyde phosphate dehydrogenase; Rossi et al., 1995, Biochem. J. 308: 455-463). The vector plasmid (pBluescript) was used as a non-specific hybridization control. Following hybridization, the filters were visualized by autoradiography and the radioactivity was quantitated by MDP analysis. The values are expressed as arbitrary units obtained by comparing transcription rates to control levels.

Protein Synthesis

K562 cells treated with 500 μM 2 cyclopenten-1-one for different times were pulse-labeled with L-[³⁵S]-methionine (10 μCi/10⁶ cells) for 1 hour. Cells were washed with phosphate buffered saline (PBS) and lysed with 400 μl of lysis buffer (20 nM Tris-Ci, pH 7.4, 0.1 M NaCl, 5 mM MgCl₂, 1% Nonidet P-40, 0.5% SDS) containing protease inhibitors. After cell lysis, the radioactivity incorporated into trichloroacetic acid-insoluble material was determined, and an aliquot of samples containing the same amount of radioactivity were analyzed by 10% SDS-PAGE and autoradiography. HSP70 synthesis was determined by densitometric analysis of the autoradiograms. Total protein synthesis was determined as [³⁵S]-methionine incorporation into TCA-insoluble material (Amici et al., 1993, Exp. Cell. Res. 207. 230-234).

Virus Infection and Replication

Confluent MA104 cells were washed in phosphate buffered saline (PBS) and infected with vesicular stomatitis virus (VSV), Indiana serotype (Orsay), 1 plaque forming unit (PFU)/cell). After a 1 hour incubation at 37° C., virus inocula were removed, and monolayers were washed three times with PBS and incubated with 1 ml of RPMI-1640 medium containing 2% FCS and control diluent or different concentrations of 2-cyclopenten-1-one dissolved in ethanol. VSV titers were determined using medium removed 24 hours post-infection (p.i.) by cytopathic effect 50% (CPE 50%) assay on confluent monolayers of MA104 cells in 96-well tissue culture dishes, as described in Pica et al., 1996, Antiviral Res. 29: 187-198.

VSV Protein Synthesis

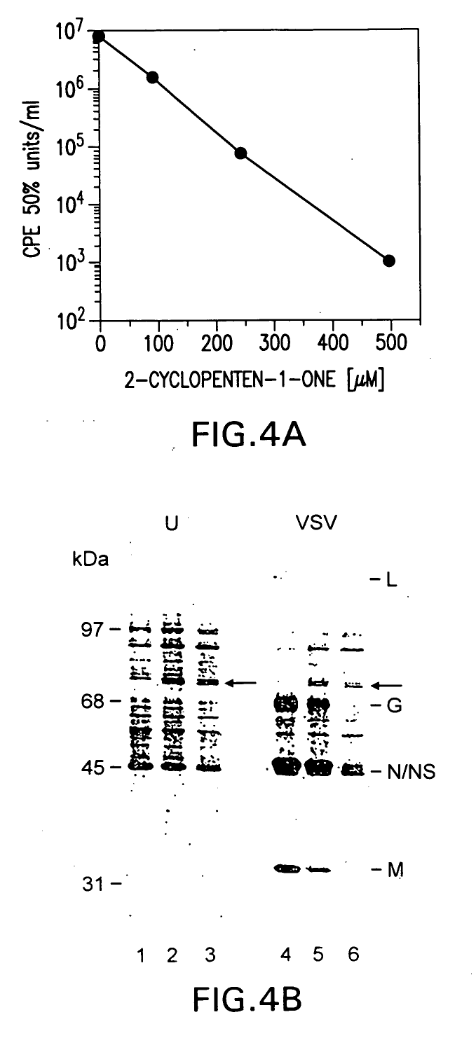

Uninfected (U) or VSV-infected (VSV) MA104 cells were treated with 250 μM of 2-cyclopenten-1-one (FIG. 4B, lanes 2 and 5), 500 μM 2-cyclopenten-1-one (FIG. 4B, lanes 3 and 6), or control diluent (FIG. 4B, lanes 1 and 4), soon after VSV infection and labeled with [³⁵S]-methionine (8 μCi/2×10⁵ cells, 1 hour pulse starting 5 h p.i.). were washed with phosphate buffered saline (PBS) and lysed with 400 μl of lysis buffer (20 nM Tris-Cl, pH 7.4, 0.1 M NaCl, 5 mM MgCl₂, 1% Nonidet P-40, 0.5% SDS) containing protease inhibitors. Equal amounts of protein were analyzed on 10% SDS-PAGE and autoradiography.

### Results

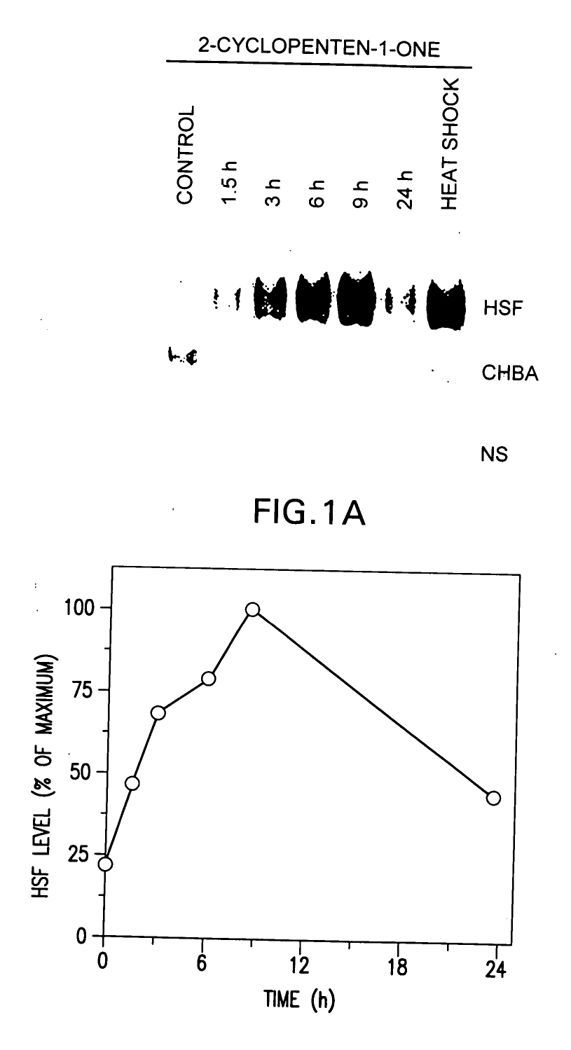

The data in FIG. 1 indicates that 2-cyclopenten-1-one induces the activation of heat shock transcription factor (HSF). As shown in FIGS. 1A and 1B, the activation of HSF by 2-cyclopenten-1-one was detected 1.5 hours after treatment, and maximum levels of HSF activation was detected 9 hours after treatment. Approximately 50% less activated HSF was detected 24 hours after 2-cyclopenten-1-one treatment than activated HSF detected 9 hours after 2-cyclopenten-1-one treatment. Thus, 2-cyclopenten-1-one induces the activation of HSF within 1.5 hours after treatment.

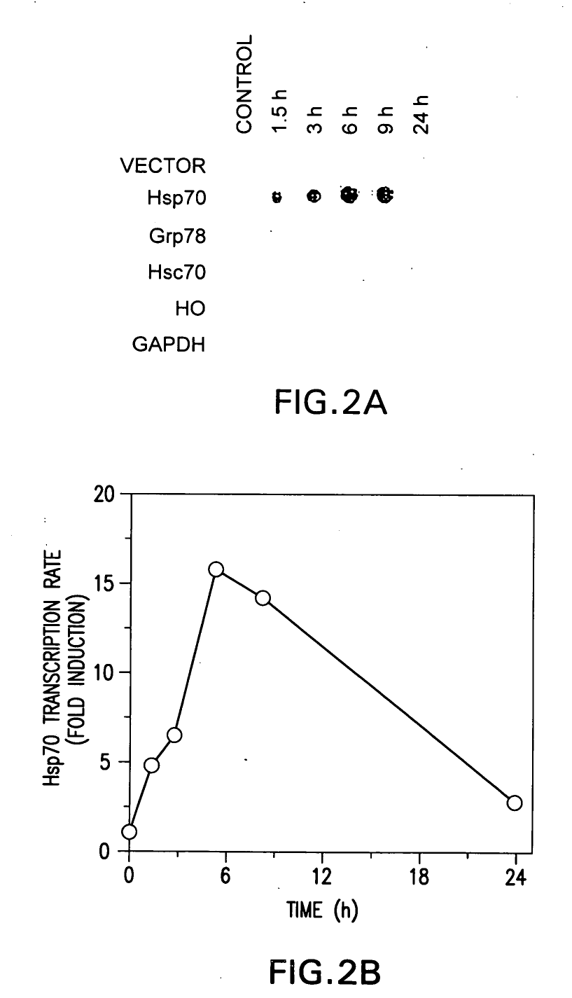

Activated HSF induces the expression of heat shock proteins (HSPs), which protect cells against a wide variety of toxic conditions, including extreme temperatures, oxidative stress, viral infection, and the exposure to heavy metals or cytotoxic drugs (Lindquist et al., 1988, Annu. Review Genet. 22: 631-677). The ability of 2-cyclopenten-1-one to induce the expression of the 70 kDa heat shock protein (HSP70) was analyzed. The data in FIG. 2 indicates that 2-cyclopenten-1-one induces the expression of HSP70. HSP70 mRNA transcription was detected 1.5 hours after 2-cyclopenten-1-one treatment, and transcription rates were maximal by 6-9 hours after 2-cyclopenten-1-one treatment. HSP70 mRNA transcription had decreased by 24 hours after 2-cyclopenten-1-one treatment. The data in FIG. 2 also indicates that 2-cyclopenten-1-one is able to selectively activate the HSP70 gene transcription. The transcription of other stress proteins, including HSC70, glucose regulated GRP78/BiP and heme-oxygenase, were not affected by 2-cyclopenten-1-one treatment.

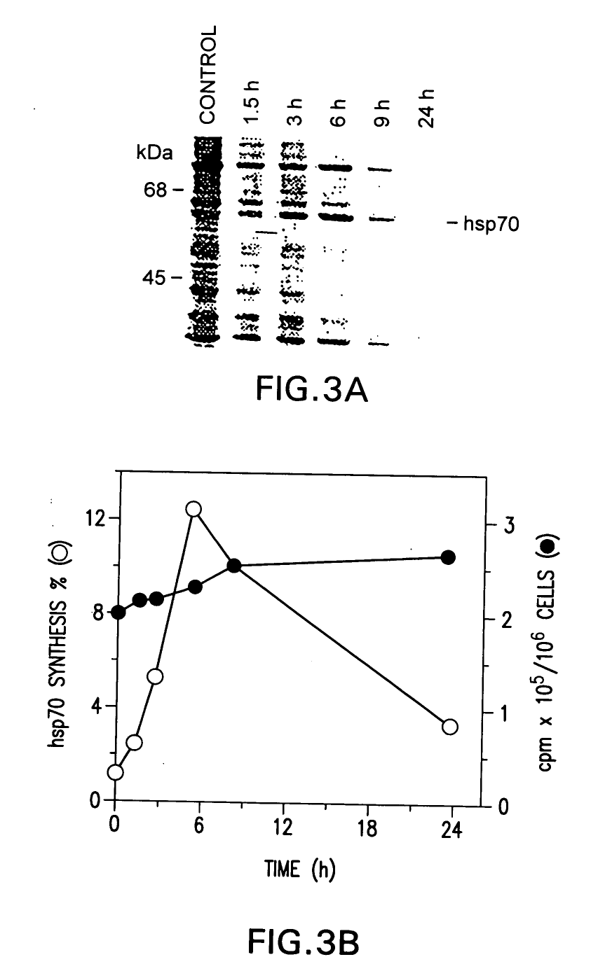

The affect of 2-cyclopenten-1-one treatment on HSP70 protein levels was analyzed by [³⁵S]-methionine incorporation. As shown in FIG. 3, 2-cyclopenten-1-one is able to selectively stimulate HSP70 protein synthesis at concentrations that do not inhibit the cellular a protein synthesis.

As shown in FIG. 4A, 2-cyclopenten-1 one was found to inhibit the production of VSV infectious particles in a dose-dependent manner. At concentrations ranging between 100 and 500 μM, 2-cyclopenten-1-one inhibits the production of VSV infectious particles from 10 to more than 1000 times with respect to the control. As shown in FIG. 4B, the inhibition of VSV infectious particle production by 2-cyclopenten-1-one is mediated by a selective block of the viral protein synthesis. Therefore, 2-cyclopenten-1-one selectively inhibits viral protein synthesis while inducing the expression of HSP70.

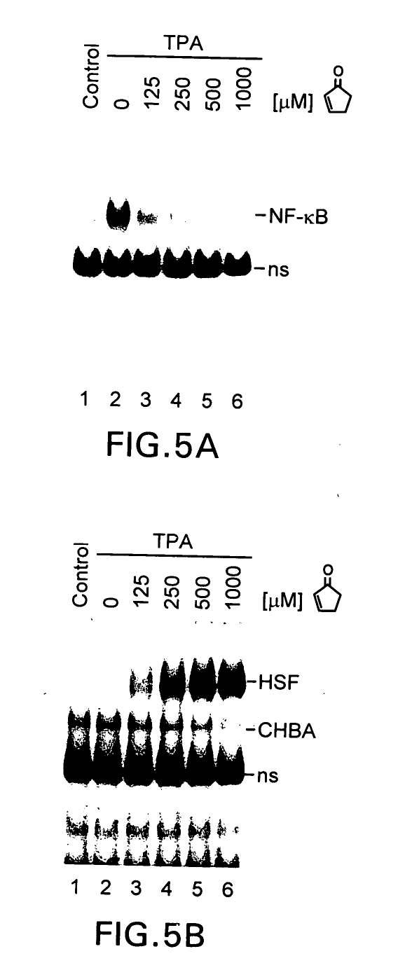

As shown in FIG. 5A, 2-cyclopenten-1-one is able to inhibit NF-κB activation by TPA at a concentration as low as 125 μM. At a concentration of 500 μM of 2-cyclopenten-1-one, NF-κB activation is not detectable (FIG. 5A). In the same samples, HSF activation is detectable in cells treated with 125 μM 2-cyclopenten-1-one (FIG. 5B). Thus, NF-κB activation appears to inversely correlate with HSF activation upon 2-cyclopenten-1-one treatment.

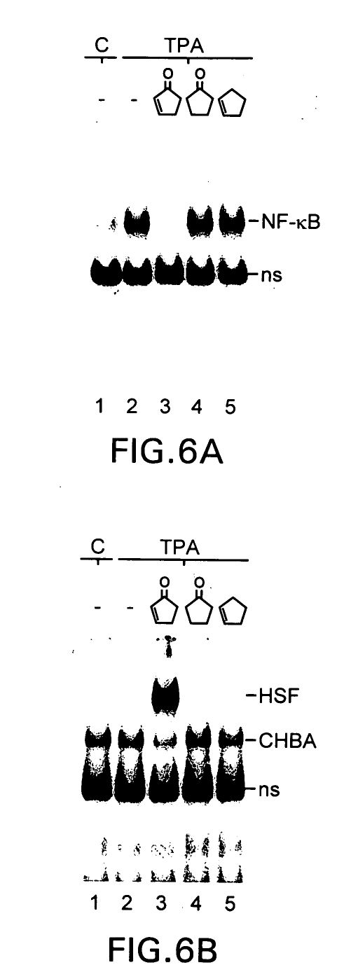

To further study the structure-activity relationship of the chemical structure needed to activate HSF and inhibit NF-κB activation, Jurkat cells were treated with 500 μM of 2-cyclopenten-1-one, 500 μM of cyclopentanone, or 500 μM of cyclopentene for 1 hour followed by TPA stimulation. After three hours, whole cell extracts were prepared and HSF and NF-κB activation were determined by EMSA. As shown in FIG. 6A, only 2-cyclopenten-1-one inhibits TPA-induced NF-κB activation (lane 3); cyclopentanone (lane 4) and cyclopentene (lane 5) do not inhibit NF-κB activation. Further, as shown in FIG. 6B, HSF activation was only detected in the sample of 2-cyclopenten-1-one treated cells (lane 2); HSF activation was not detected in samples of cyclopentanone (lane 4) and cyclopentene (lane 5) treated cells. These results demonstrate that the α,β-unsaturated carbonyl group is the key structure triggering HSF activation and its presence is necessary to inhibit NF-κB activation, in cyclopentyl or prostaglandin type compounds.

### 7. EXAMPLE

### 3,4-Dichloro-Iso-Coumarine Induces HSF Activation, Inhibits Viral Replication, and Inhibits NF-κB Activation

The following example demonstrates the ability of 3,4-dichloro-iso-coumarine (DCIC) to induce HSP70 transcription, to inhibit viral replication, and to inhibit NF-κB activation in vitro.

### Materials & Methods

Cell Culture

Monolayers of monkey kidney cells (MA104 cells), and Jurkat cells were grown in RPMI 1640 medium supplemented with 10% fetal calf serum and antibiotics at 37° C. in a 5% CO₂ humidified atmosphere (Amici et al., 1995, Cancer Research 55: 14452-4457).

Electrophoretic Mobility Shift Assay

Jurkat cells were treated with 5 μM of DCIC dissolved in ethanol at 37° C. or control diluent (negative control). Alternatively, Jurkat cells were treated with different concentrations of DCIC, Nα-tosyl-lysine-chloromethylketone (TLCK), or tosyl-L-phenylalanine-chloromethylketone (TPCK) for 1 hour and then were stimulated with TPA (25 ng/ml). At different times or three hours after treatment, whole cell extracts were prepared and analyzed by EMSA (Electrophoretic Mobility Shift Assay) as described in Amici et al., 1995, supra. Briefly, extracts (10 μg/sample) were mixed with 0.1 ng of a ³²P-NF-κB element or ³²P—HSE oligonucleotide and 0.5 μg of poly(dI-dC) (Pharmacia Biotech Inc.) in 25 μl of binding buffer (10 nM Tris-Cl, pH 7.8, 50 mM NaCl, 1 mM EDTA, 0.5 mM dithiothreitol, 5% glycerol). After a 20 minute incubation at room temperature, HSF-HSE or NF-κB-DNA-complexes were analyzed by nondenaturing 4% polyacrylamide gel electrophoresis and autoradiography. The amount of shifted HSE probe and NF-κB probe, an indicator of HSF DNA-binding activity and NF-κB activity, respectively, were quantitated by Molecular Dynamics Phospholmager (MDP) analysis.

Transcription Run-On Assay

In vitro run-on transcription reactions were performed performed in isolated Jurkat nuclei as described in Banerji et al, 1984, Mol. Cell. Biol. 5: 2437-2448. ³²P-labeled RNA was hybridized to nitrocellulose filters containing plasmids for the following human genes: hsp7O (pH 2.3; Wu et al., 1985, Mol. Cell. Biol. 5: 330-341); grp78/BiP (glucose-regulated 78 protein) (pHG 23.1; Amici et al., 1992, Proc. Natl. Acad. Sci. USA 89, 6227-6231); hsc7O (heat shock cognate70) (pHA 7.6; Amici et al., 1992, Proc. Natl. Acad. Sci. USA 89, 6227-6231); HO (heme oxygenase) (HO clone 2/10; Rossi et al., 1995, Biochem. J. 308: 455-463); and GAPDH (rat glyceraldehyde phosphate dehydrogenase; Rossi et al., 1995, Biochem. J. 308: 455-463). The vector plasmid (pBluescript) was used as a non-specific hybridization control. Following hybridization, the filters were visualized by autoradiography and the radioactivity was quantitated by MDP analysis. The values are expressed as arbitrary units obtained by comparing transcription rates to control levels.

Virus Infection and Replication

Confluent MA104 cells were washed in phosphate buffered saline (PBS) and infected with vesicular stomatitis virus (VSV), Indiana serotype (Orsay), 1 plaque forming unit (PFU)/cell). After a 1 hour incubation at 37° C., virus inocula were removed, and monolayers were washed three times with PBS and incubated with 1 ml of RPMI-1640 medium containing 2% FCS and control diluent or different concentrations of 2-cyclopenten-1-one dissolved in ethanol. VSV titers were determined using medium removed 24 hours post-infection (p.i.) by cytopathic effect 50% (CPE 50%) assay on confluent monolayers of MA104 cells in 96-well tissue culture dishes, as described in Pica et al., 1996, Antiviral Res. 29: 187-198.

VSV Protein Synthesis

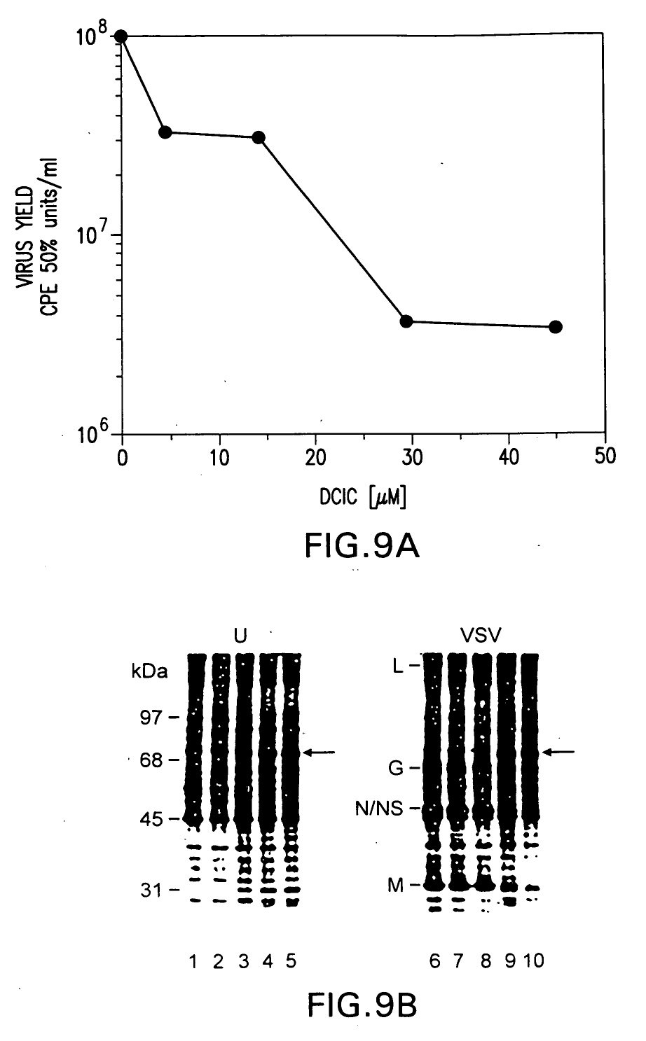

Uninfected (U) or VSV-infected (VSV) MA104 cells were treated with 5 μM of DCIC (FIG. 9B, lanes 2 and 7), 15 μM of DCIC (FIG. 9B, lanes 3 and 8), 30 μM of DCIC (FIG. 9B, lanes 4 and 9), 45 μM of DCIC (FIG. 9B, lanes 5 and 10), or control diluent (FIG. 9B, lanes 1 and 6), soon after VSV infection and labeled with [³⁵S]-methionine (8 μCi/2×10⁵ cells, 1 hour pulse starting 5 h p.i.). were washed with phosphate buffered saline (PBS) and lysed with 400 μl of lysis buffer (20 nM Tris-Cl, pH 7.4, 0.1 M NaCl, 5 mM MgCl₂, 1% Nonidet P-40, 0.5% SDS) containing protease inhibitors. Equal amounts of protein were analyzed on 10% SDS-PAGE and autoradiography.

### Results

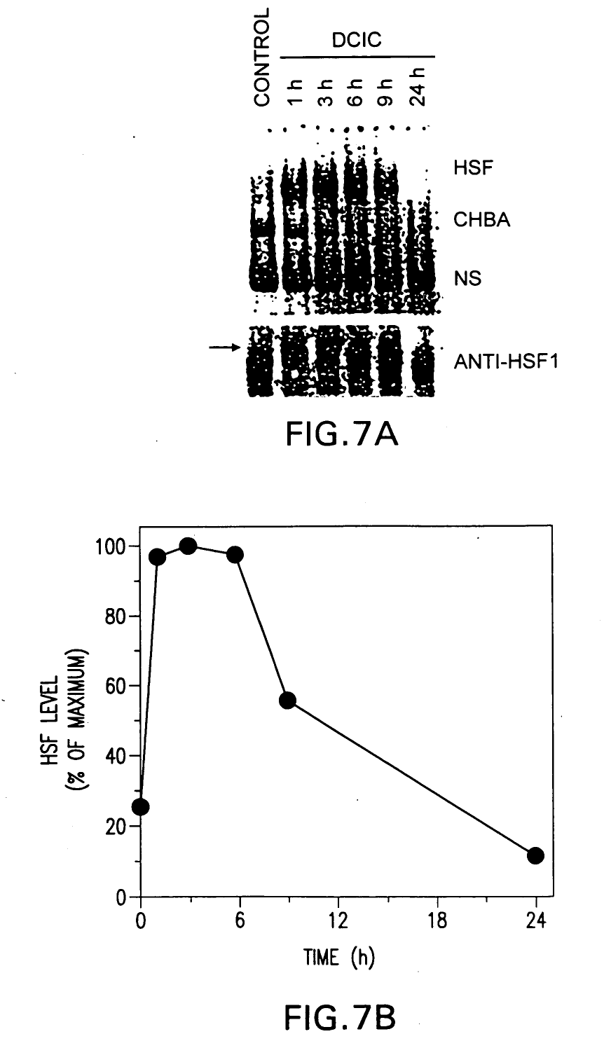

The data in FIG. 7 indicates that DCIC induces the activation of heat shock transcription factor (HSF). As shown in FIGS. 7A and 7B, the activation of HSF by DCIC was detected 1 hour after treatment, and maximum levels of HSF activation was detected 3 hours after treatment. The activation of HSF is prolonged for 12 hours following the initiation of DCIC treatment. Thus, DCIC induces the activation of HSF within 1 hour after treatment.

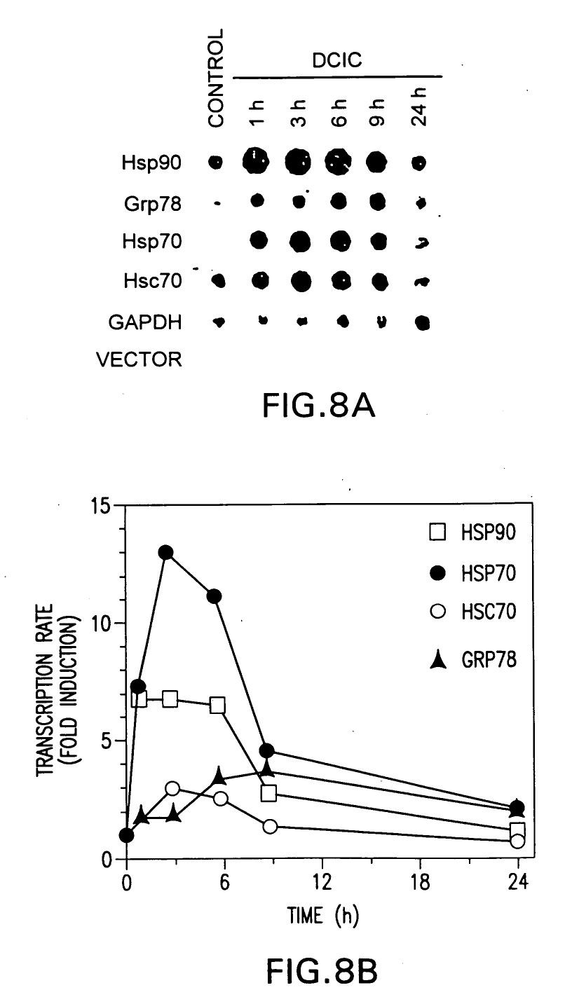

Activated HSF induces the expression of heat shock proteins (HSPs), which protect cells against a wide variety of toxic conditions, including extreme temperatures, oxidative stress, viral infection, and the exposure to heavy metals or cytotoxic drugs (Lindquist et al., 1988, Annu. Review Genet. 22: 631-677). The ability of DCIC to induce the expression of stress proteins (HSP90, Grp78, HSC70, and HSP70) was analyzed. The data in FIG. 8 indicates that DCIC selectively induces the expression of HSP90 and HSP70. HSP90 and HSP70 mRNA transcription were detected 1 hour after DCIC treatment, and transcription rates were maximal by about 3 hours after DCIC treatment. HSP90 and HSP70 mRNA transcription had decreased by 24 hours after DCIC treatment. Thus, DCIC selectively induces the transcription of HSP90 and HSP70.

To determine the ability of DCIC to inhibit VSV replication, the production of VSV infectious particles was analyzed. As shown in FIG. 9A, DCIC was found to inhibit the production of VSV infectious particles in a dose-dependent manner. At concentrations ranging between 5 and 45 μM, DCIC inhibits the production of VSV infectious particles from 50% to more than 98% with respect to the control. As shown in FIG. 9B, the inhibition of VSV infectious particle production by DCIC is mediated by a selective block of the viral protein synthesis. Therefore, DCIC selectively inhibits viral protein synthesis while inducing the expression of HSP90 and HSP70.

The affect of serine proteases other than DCIC on HSF activation were analyzed. Jurkat cells were incubated with the compounds listed in Table 1 (infra) or reference diluent at different concentrations for 1 hour and then were stimulated with TPA (25 ng/ml). After 1 hour at 37° C. the whole-cell extracts were prepared and subjected to EMSA to determine NF-κB and HSF activation. The levels of binding-DNA activity of NF-κB were quantified with Molecular Dynamics Phosphorlmager analysis. Table 1 shows that 4 serine protease inhibitors, tosyl-L-phenylalanine-chloromethylketone (TPCK), N_{α}-tosyl-lysine-chloromethylketone (TLCK), N-acethyl-DL-phenylalanine-β-napthylester (APNE), and N-benzoyl-L-thyroxine-ethylester (BTEE), activate HSF at the same concentrations at which they inhibited NF-κB, 90% inhibitory concentration (IC₉₀).

TABLE 1

| Inhibitor of protease | NF-κB inhibition IC₉₀ (μM) | Activation of HSF |
|---|---|---|
| (DCIC) | 5.5 | + |
| (TPCK) | 12 | + |
| (TLCK) | 135 | + |
| (APNE) | 300 | + |
| (BTEE) | 400 | + |

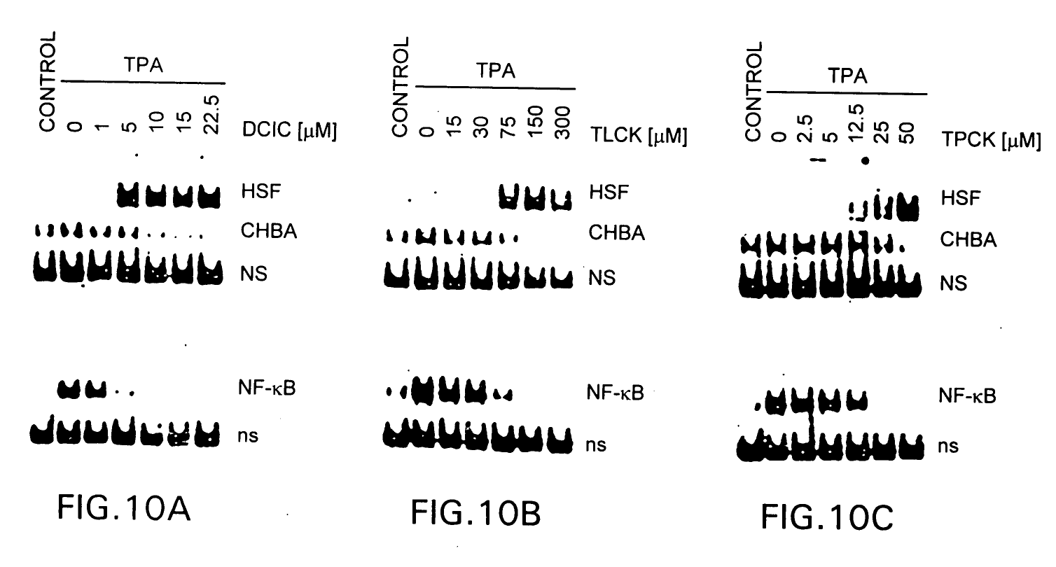

The concentration of DCIC, TLCK or TPCK needed to activate HSF and to inhibit NF-κB activation was analyzed by EMSA. As shown in FIG. 10A, DCIC was found to activate HSF and to inhibit NF-κB activation at concentrations ranging between 5-10 μM. As shown in FIG. 10B, TLCK was found to activate HSF and to inhibit NF-κB activation at concentrations ranging between 30-75 μM. As shown in FIG. 10C, TPCK was found to activate HSF and to inhibit NF-κB activation at concentrations ranging between 12.5-25 μM. Therefore, DCIC, TLCK and TPCK activate HSF at the concentration that inhibit NF-κB activation by TPA.

### 8. EXAMPLE

### Δ¹²-Prostaglandin J2 is a Potent Inhibitor of Influenza A Virus Replication In Vivo

The following example demonstrates that Δ¹²-PGJ₂ (9-deoxy-Δ⁹,Δ¹²-13,14-dihydro-PGD₂), a natural cyclopentenone metabolite of PGD₂ physiologically present in human body fluids, is a potent inhibitor of influenza A virus replication in vitro and in vivo.

### Materials & Methods

Cell Culture

Madin-Darby canine kidney (MDCK) cells were grown at 37° C. in RPMI-1640 medium, supplemented with 5% fetal calf serum (FCS; Gibco BRL) and antibiotics.

Cell viability was determined by the dye exclusion technique and by the 3-(4,5-dimethylthiazol-2-yl)-2,5-diphenyltetrazolium bromide (MTT) assay, as previously described (Shigeta et al., 1997, Antimicrob. Agents Chemother. 41: 1423-1427). For MTT assay uninfected MDCK cells were treated with 6 μg/ml Δ¹²-PGJ₂ or ethanol diluent for 24 hours. After this time, 10 μl of a 0.5% MTT solution in PBS was added to the monolayers and the mixture was incubated for 2 hours at 37° C. Reduced MTT (formazan) was extracted from cells by adding 100 μl of acidic isopropanol containing 10% Triton X-100, and formazan absorbance was measured in an ELISA microplate reader at two different wavelengths (540 and 690 nm).

Viral Infection and Replication

Influenza A virus A/PR8/34 (H1N1) (PR8 virus) (Santoro et al., 1988, Arch. Virol. 99: 89-100) was grown in the allantoic cavity of 10-day-old embryonated eggs. Virus titers were determined by hemagglutinin titration, according to standard procedures. One hemagglutinating unit (HAU) corresponded to 10⁶ PFU in this model. Confluent MDCK monolayers were infected with PR8 virus (5 HAU/10⁵ cells) for 1 hour at 37° C. Subsequently, viral inoculum was removed and cells were treated with different concentrations of Δ¹²-PGJ₂ (Cayman-Chemical Co.) or ethanol-diluent, which did not affect cell metabolism or virus replication. Viral yields were determined 24 or 72 hours post infection (p.i.). Virus titers were determined in triplicate samples by both HAU and cytopathic effect 50% (CPE5O %) assay on confluent MDCK monolayers, as previously described (Pica et al., 1993, Antiviral Res. 20: 193-208).

³H-Thymidine and ³H-Uridine Incorporation Assays

Uninfected or PR8-infected MDCK cells were treated with Δ¹²-PGJ₂ (6 μg/ml) or control diluent soon after a 1 hour adsorption period, and labeled for the following 24 hours with [³H]thymidine or [³H]uridine (5 μCi/10⁵ cells) for DNA and RNA synthesis, respectively. The radioactivity incorporated into trichloroacetic acid-soluble (uptake) and -insoluble (incorporation) material was determined as described (Rozera et al., 1996, J. Clin. Invest. 97: 1795-1803).

Protein Synthesis

PR8-infected MDCK monolayers were treated with Δ¹²-PGJ₂ (6 μg/ml) or ethanol-diluent after a 1 hour adsorption period, and labeled with [³⁵S]methionine (5 μCi/10⁵ cells) for the following 24 hours. Uninfected cells were treated identically. After this time, the amount of radioactivity incorporated into proteins was quantified and samples containing equal amounts of radioactivity were analyzed by SDS-polyacrylamide gel electrophoresis (SDS-PAGE) in a vertical slab gel apparatus (3% stacking gel, 10% resolving gel) and processed for autoradiography as described (Rossi et al., 1997, J. Biol. Chem. 271: 32192-32196). Molecular weights were calculated by using Bio-Rad Mᵣ markers. For immunoblot (IB) analysis, equal amounts of protein from each sample were separated by SDS-PAGE and blotted onto nitrocellulose, and filters were incubated with monoclonal anti-HSP70 antibodies (Rossi et al., 1997, supra). Virus proteins were identified by IB analysis using a polyclonal anti-WSN virus antiserum which recognizes PR8 virus HA, NP and M1 proteins (Basler et al., 1999, J. Virol. 73: 8095-8103), and a monoclonal anti-HA antibody (kindly supplied by E. Rodriguez-Boulan, Cornell University, NY).

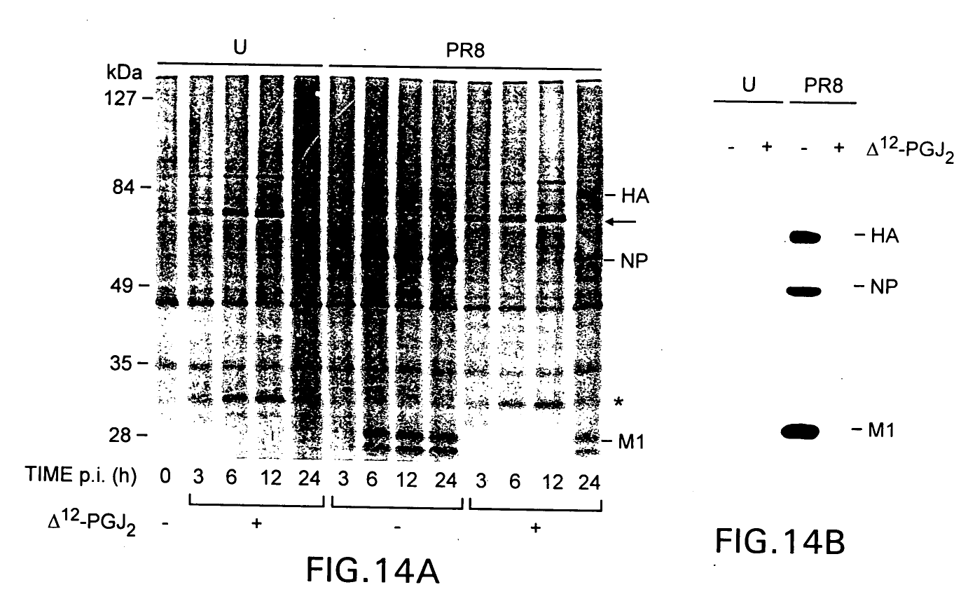

To investigate the kinetics of HSP70 and virus protein synthesis, PR8-infected or mock-infected cells were treated with Δ¹²-PGJ2 (6 μg/ml) or ethanol-diluent after a 1 hour adsorption period, and labeled with [³⁵S]methionine (10 μCi/10⁵ cells, 45 min pulse) at different times p.i. Samples containing the same amount of radioactivity were processed for SDS-PAGE and autoradiography. Alternatively, equal amounts of protein from unlabeled uninfected or PR8-infected cells at 24 hours p.i. were processed for IB analysis using polyclonal anti-WSN virus antiserum (FIG. 14B) or monoclonal anti-HA antibodies (data not shown).

In Vivo Infection

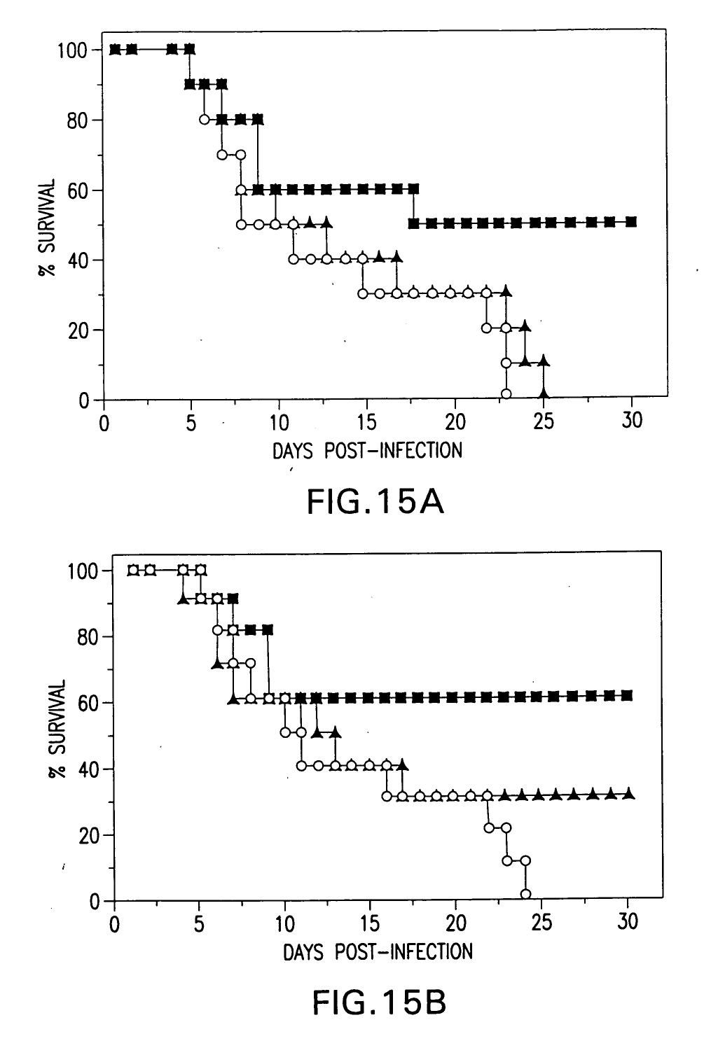

200 4-week old Balb/c male mice were inoculated intranasally (i.n.) with 100 μl PR8-virus suspension (12.5 HAU/ml) under light ether anesthesia, and, 3 hours after inoculation, were randomly divided in groups of 10 or 20 and injected intraperitoneally (i.p.) with 100 μl sterile saline-solution containing 10% ethanol or Δ¹²-PGJ₂-ethanolic solution, according to different schedules (FIG. 15). Mice were examined daily for survival for the following 4 months. Survival curves were compared using the Sign test (Santoro et al., 1988, supra) and a values <0.05 were considered significant.

### Results

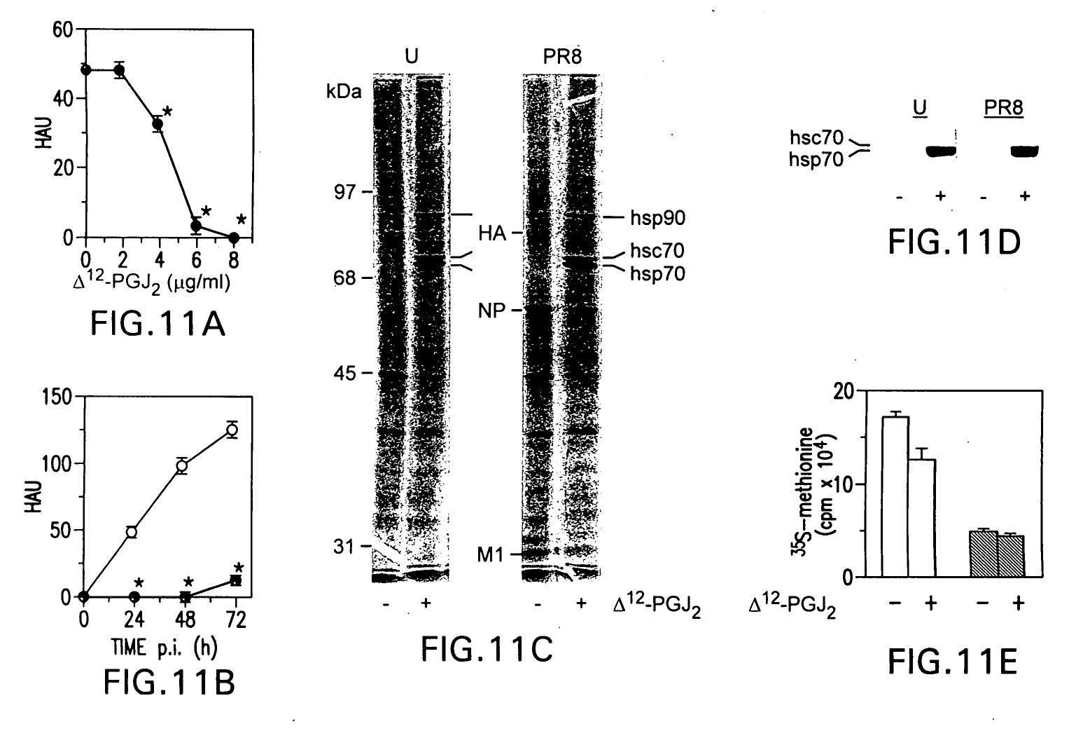

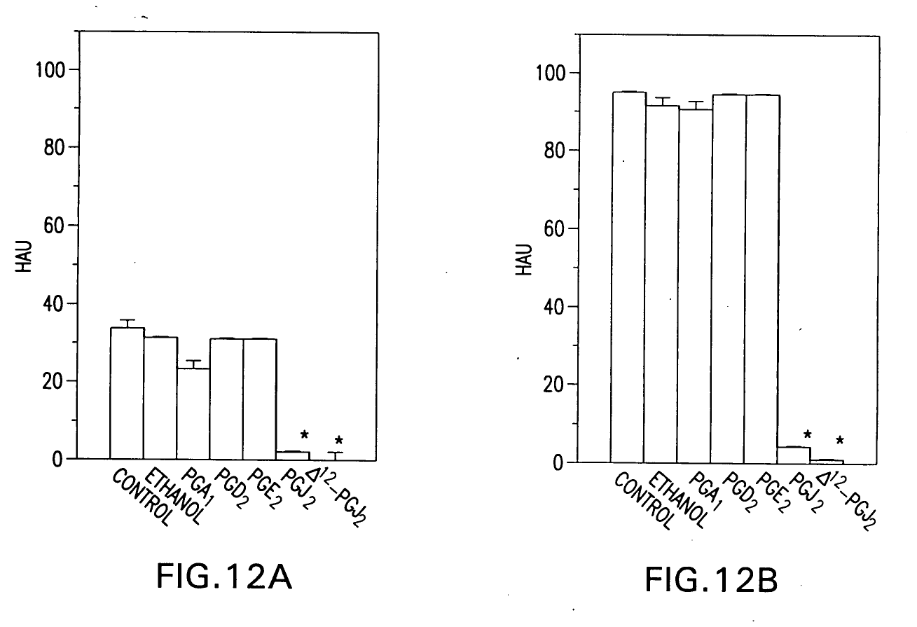

The data in FIG. 11A indicates that Δ¹²-PGJ₂ inhibit PR8 viral production. In particular, FIG. 11A demonstrates that Δ¹²-PGJ₂ reduces PR8 production dose-dependently; an inhibition greater than 95% of control was obtained at the concentration of 6 μg/ml, whereas virus yield was not detectable at higher concentrations. FIG. 11B demonstrates that the antiviral activity of 6 μg/ml of Δ¹²-PGJ₂ is sustained for a period of at least 72 hours p.i. Treatment of PR8 virus (1 HAU/10 cells)-infected MDCK cells with 6 μg/ml Δ¹²-PGJ₂ after the 1 hour adsorption period caused the expected decrease in HAU (Control: 16±0; Δ¹²-PGJ₂-treated: 0 HAU), and it was effective in reducing infectious virus titers by more than 99% of control (Control: 3.6±0.4×10⁴ Δ¹²-PGJ₂-treated: 3.2+0.6×10¹ CPE5O % units/ml). When the antiviral activity of Δ¹²-PGJ₂ was compared with the effect of other prostaglandins, Δ¹²-PGJ₂ was found to be the most effective cyclopentenone prostaglandin. Two different cyclopentenone prostaglandins, PGA, and PGJ₂, inhibited PR8 replication in vitro, though to a minor extent as compared to Δ¹²-PGJ₂. Prostaglandin A₁ (PGA₁), which possesses antiviral activity against several RNA viruses (Pica et al., 1993, supra), only modestly and transiently inhibited PR8 replication at the concentration of 6 μg/ml; however, at higher concentrations (10 μg/ml), PGA, was effective in decreasing PR8 virus yield by more than 90% of control up to 48 hours p.i. (data not shown). Non-cyclopentenone prostaglandins of the E and D type, which do not activate HSF and are not able to induce HSP70 synthesis (Rossi et al., 1997, J. Biol. Chem. 271: 32192-32196), and do not affect PR8 virus replication (FIG. 12).

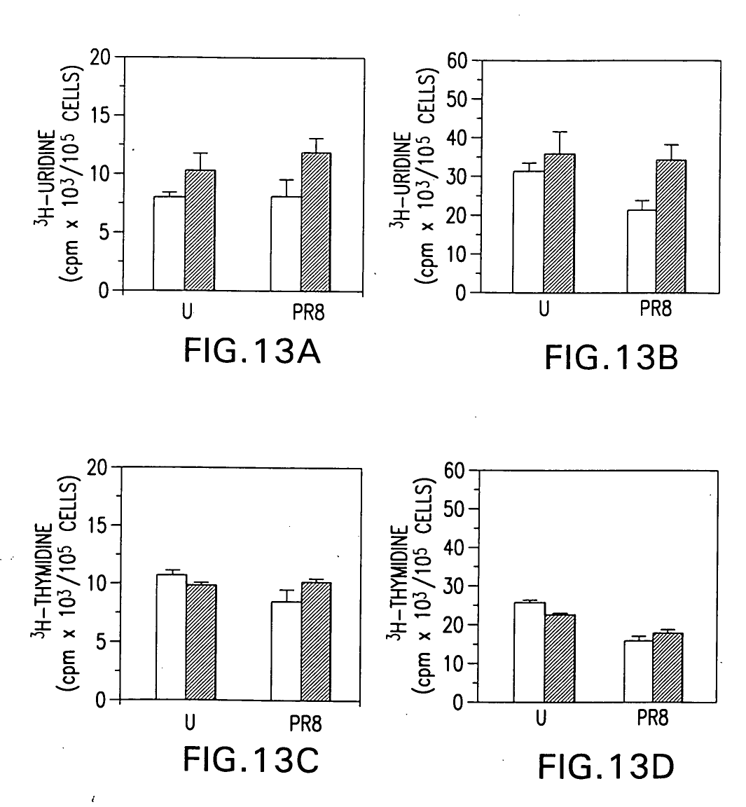

The effect of Δ¹²-PGJ₂ on cell viability of uninfected cells was analyzed using the MTT assay. The results from quadruplicate samples show that Δ¹²-PGJ₂ did not affect cell viability (ethanol control: 292±21; Δ¹²-PGJ₂-treated: 332+44). Moreover, treatment with Δ¹²-PGJ₂ did not inhibit nucleic acid synthesis in MDCK cells. As shown in FIG. 13, Δ¹²-PGJ₂-treatment did not inhibit either the uptake of precursors or DNA and RNA synthesis in both uninfected and PR8-infected cells. Δ¹²-PGJ₂-treatment actually prevented the virus-induced inhibition of cellular RNA synthesis (FIG. 13B).

To investigate the effect of Δ¹²-PGJ₂ on cellular and viral protein synthesis, PR8-infected MDCK monolayers treated with Δ¹²-PGJ₂ (6 μg/ml) or ethanol-diluent were analyzed by SDS-PAGE. In uninfected cells, Δ¹²-PGJ₂ caused a modest reduction of protein synthesis (FIG. 11E) and did not alter the overall electrophoretic profile of cellular proteins, whereas it markedly induced the synthesis of two polypeptides of 70 and 72 kDa Mᵣ respectively, which were identified as the constitutive (HSC70) and the inducible (HSP70) form of heat-shock protein HSP70 by immunoblot analysis (FIGS. 11C and 11D). Synthesis of HSP90 was also enhanced. PR8-infection caused a decrease in protein synthesis (FIG. 11E) and did not induce HSP70 synthesis in MDCK cells (FIGS. 11C and 11D). Δ¹²-PGJ₂-treatment caused a dramatic reduction of PR8 protein synthesis, which was associated with the synthesis of high levels of HSC70 and HSP70 (FIG. 1C). NP synthesis appeared to be reduced by a lesser extent as compared to the other viral proteins. As shown in FIG. 14A, HSP70 synthesis was detectable 3 hours after Δ¹²-PGJ₂-treatment and continued to be detectable for at least 12 hours in both uninfected and PR8-infected MDCK cells. As previously shown for other negative strand RNA viruses (Santoro et al., 1988, supra), PR8 virus protein synthesis was inhibited as long as HSP70 was being synthesized by the host cell (FIG. 14A), and viral proteins were not detectable by immunoblot (IB) analysis in Δ¹²-PGJ₂-treated cells at 24 hours p.i. (FIG. 14B). Thus, these results indicate that Δ¹²-PGJ₂ inhibits viral protein synthesis while inducing the protein synthesis of HSP70.

A role for HSP70 as the cellular mediator interfering with viral protein synthesis during negative-strand RNA virus infection was suggested, as different HSP70 inducers, including sodium arsenite, cadmium, azetidine and heat shock, all selectively inhibit viral protein synthesis (Santoro et al., 1997, supra); conversely, cyPG-treatment has no effect on viral protein synthesis in cells lacking the ability to synthesize HSP70 or during infection with viruses that shut-off HSP70 synthesis (Centers for Disease Control and Prevention. 1996. Prevention and Control of Influenza: Recommendations of the Advisory Committee on Immunization Practices. Morbid. Mortal. Weekly Rep. 45(RR-5): 1-24, Santoro et al., 1997, supra, Superti et al., 1998, J. Infect. Dis. 178: 564-568). The mechanism by which HSP70 can interfere with viral protein synthesis remains to be elucidated. HSP70 could directly interact with the nascent viral polypeptides, causing a translational block. Alternatively, it was hypothesized that HSP70 and virus messages could possess similar mechanisms for preferential translation and compete with each other (Santoro et al., 1997, supra).

To evaluate whether Δ¹²-PGJ₂ could also be effective in controlling influenza A-infection in vivo a series of experiments using Balb/c mice as recipients for PR8-virus were performed. Depending on the dose, PR8-virus intranasal (i.n.) inoculation produces a damaging infection of the lungs, highly lethal to the animals; infection with 1 HAU PR8-virus/mouse results in 100% death of 4 week-old Balb/c mice in the first month p.i. (Santoro et al., 1988, supra). As expected, 100% of PR8-infected animals treated with 100 μl sterile saline containing 10% ethanol (control animals) were dead by day 24 p.i. Ethanol-diluent did not significantly affect mouse survival after PR8-infection (Santoro et al., 1988, supra). As shown in FIG. 15A, treatment of PR8-infected mice with Δ¹²-PGJ₂ 1 μg/day/mouse for 7 days had no effect on mouse survival. However, treatment with Δ¹²-PGJ₂ 5 μg/day/mouse for 7 days resulted in a significant increase in mice survival (50% on day 25 p.i.; FIG. 15A). As shown in FIG. 15B, treatment with 5 μg/day/mouse Δ¹²-PGJ₂ on days 0, 2 and 4 after PR8-infection was less effective, resulting in the survival of approximately 30% of the animals, as compared to 60% when Δ¹²-PGJ₂ was administered daily for 7 days. Mice that survived to day 25 p.i. did not show any sign of disease for the following 3 months and were considered cured. As compared to mock-infected controls, PR8-infected mice showed a significative reduction in body weight at 7 days p.i. (Control: 19.73±0.61; PR8-infected: 15.97±0.98 g; P=0.001). Loss of body weight at 7 days p.i. was significantly decreased in mice that received a 7-day Δ¹²-PGJ₂-treatment (5 μg/day/mouse) (PR8-infected: 15.97±0.98; Δ¹²-PGJ₂-treated, PR8-infected: 18.25±0.68 g; P=0.001).

To determine the effect of Δ¹²-PGJ₂ on the virus titer in the lung, ten 4 week-old Balb/c mice were inoculated with 1 HAU of PR8 virus/mouse and treated daily with Δ¹²-PGJ₂ (10 μg/mouse/day i.p., n=5) or ethanol diluent (n=5). Four days after virus infection, mice were sacrificed and the virus titer in the lungs was determined by CPE50% assay on MDCK cells, as described previously (Santoro et al., 1988, supra). Δ¹²-PGJ₂-treatment was found to cause a decrease in virus titer in the lungs (Control: 6.44±2.77×10⁴; Δ¹²-PGJ₂-treated: 0.07±0.03×10⁴ CPE50% units/gr of lung tissue; p<0.001), indicating a direct action of Δ¹²-PGJ₂ on virus replication in this organ. Animals treated with the higher dose of Δ¹²-PGJ2 (10 μg/day/mouse i.p., for 4 days) did not show any sign of toxicity; in fact, Δ¹²-PGJ₂-treatment reduced the loss of body weight in infected animals (data not shown). Thus, administration of Δ¹²-PGJ₂ to PR8-infected mice is effective in protecting mice from viral infection and decreasing viral titers in the lung. These results suggest a therapeutic use of cyclopentenone prostanoids or prostanoid-derived molecules during clinical complications of influenza virus infection.

1. A method of treating or preventing virus replication and related disorders in an animal comprising administering to the animal in which such treatment or prevention is desired a therapeutically effective amount of a compound with a cyclopentenone ring structure wherein the compound is not PGD₂, PGA₂ 15-deoxy-13,14-dihydroprostaglandin J₂, Δ¹²-13, 14-dihydro-PGD₂ or the compound depicted below 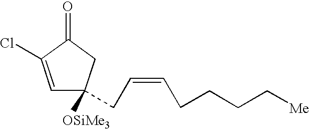
and wherein the compound has a cylcopentenone ring structure and has an aliphatic side chain at position 4 or 5 and lacks an aliphatic side chain at the position 4 or 5 not containing the aliphatic chain, or has a cylcopentenone ring structure and lacks an aliphatic side chain at both position 4 and 5.

2. The method of claim 1 wherein the virus is human immunodeficiency virus, influenza, herpesvirus, hepatitis B virus, hepatitis C virus, human T-cell lymphotrophic virus type I, human T-cell lymphotrophic virus type II, lassa fever virus, morbillivirus virus, human respiratory syncytial virus, mumps, pneumovirus, adenovirus, hantavirus, cornavirus, Ebola virus, yellow fever virus, Japanese encephalitis virus, papillomavirues), rhinovirus, enterovirus, hepatitis A virus, poxvirus, rotavirus, rubella virus or rabies virus.

3. A method of treating or preventing inflammation and related disorders in an animal comprising administering to the animal in which such treatment or prevention is desired a therapeutically effective amount of a compound with a cyclopentenone ring structure, wherein the compound is not PGD₂, PGA₂ 15-deoxy-13,14-dihydroprostaglandin J₂, Δ¹²-13, 14-dihydro-PGD₂, or the compound depicted below 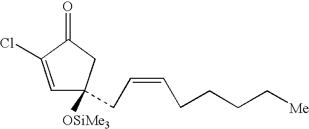
and wherein the compound has a cylcopentenone ring structure and has an aliphatic side chain at position 4 or 5 and lacks an aliphatic side chain at the position 4 or 5 not containing the aliphatic chain, or has a cylcopentenone ring structure and lacks an aliphatic side chain at both position 4 and 5.

4. A method of treating or preventing cancer and related disorders in an animal comprising administering to the animal in which such treatment or prevention is desired a therapeutically effective amount of a compound with a cyclopentenone ring structure, wherein the compound is not PGD₂, PGA₂, 15-deoxy-13,14-dihydroprostaglandin J₂, Δ¹²-13, 14-dihydro-PGD₂ or the compound depicted below. 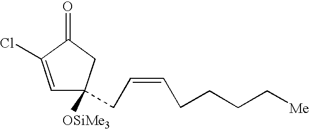
and wherein the compound has a cylcopentenone ring structure and has an aliphatic side chain at position 4 or 5 and lacks an aliphatic side chain at the position 4 or 5 not containing the aliphatic chain, or has a cylcopentenone ring structure and lacks an aliphatic side chain at both position 4 and 5.

5. A method of inducing cytoprotective responses in a human, comprising administering to a human in which such treatment is desired a therapeutically effective amount of a compound with a cyclopentenone ring structure that induces the expression of one or more heat shock proteins and wherein the compound has a cylcopentenone ring structure and has an aliphatic side chain at position 4 or 5 and lacks an aliphatic side chain at the position 4 or 5 not containing the aliphatic chain, or has a cylcopentenone ring structure and lacks an aliphatic side chain at both position 4 and 5.

6. A method of inhibiting NF-κB activation in a human, comprising administering to a human in which such treatment is desired a therapeutically effective amount of a compound with a cyclopentenone ring structure that downregulates or inhibits NF-κB activity and wherein the compound has a cylcopentenone ring structure and has an aliphatic side chain at position 4 or 5 and lacks an aliphatic side chain at the position 4 or 5 not containing the aliphatic chain, or has a cylcopentenone ring structure and lacks an aliphatic side chain at both position 4 and 5.

7. Cancelled

8. The method of claim 1, 3, 4, 5 or 6 wherein the compound is PGJ₂, 15-deoxy Δ^{12,12}-PGJ₂ or PGA₁.

9. The method of claim 5 or 6 wherein the compound is PGA₁, PGA₂, PGA₂, 16,16-dimethyl-PGA₂, PGD₂, 9-deoxy-Δ⁹,Δ¹²-13,14-dihydro-PGD₂ (Δ¹²-PGJ₂), PGJ₂, 15-deoxy Δ¹²⁻¹⁴-PGJ₂ or 2-cyclopenten-1-one.

10. Cancelled.

11. Cancelled.

12. The method of claim 5 wherein at least one of the heat shock proteins induced is HSP70.

13. The method of claim 5 or 6 wherein the human has an infectious disease.

14. The method of claim 5 or 6 wherein the human has an immune disorder.

15. The method of claim 5 or 6 wherein the human has cancer.

16. The method of claim 5 or 6 wherein the human has an inflammatory disorder.

17. The method claim 5 or 6 wherein the human has an HIV infection, an influenza virus infection, a herpesvirus infection, a hepatitis B virus infection, of a hepatitis C virus infection, human T-cell lymphotrophic virus type I, human T-cell lymphotrophic virus type II, lassa fever virus, morbillivirus virus, human respiratory syncytial virus, mumps, pneumovirus, adenovirus, hantavirus, cornavirus, Ebola virus, yellow fever virus, Japanese encephalitis virus, papillomavirues), rhinovirus, enterovirus, hepatitis A virus, poxvirus, rotavirus, rubella virus or rabies virus.

18. A method of treating or preventing a viral infection in an animal in need thereof comprising:
(a) identifying a compound that induces the expression of one or more heat shock proteins and downregulates or inhibits NF-κB activation; and
(b) administering the compound to the animal.

19. A method of treating or preventing inflammation and related disorders in an animal in need thereof comprising:
(a) identifying a compound that induces the expression of one or more heat shock proteins and downregulates or inhibits NF-κB activation; and
(b) administering the compound to the animal.

20. A method of treating or preventing cancer and related disorders in an animal in need thereof comprising:
(a) identifying a compound that induces the expression of one or more heat shock proteins and downregulates or inhibits NF-κB activation; and
(b) administering the compound to the animal.

21. The method of claims 18, 19 or 20 wherein the animal is human.

22. The method of claim 5 wherein the compound further down-regulates or inhibits NF-κB activity.

23. The method of claim 22 wherein the compound is PGJ₂, 15-deoxy Δ^{12,12}-PGJ₂ or PGA₁.

24. The method of claim 22 wherein the compound is PGA₁, PGA₂, PGA₂, 16,16-dimethyl-PGA₂, PGD₂, 9-deoxy-Δ⁹,Δ¹²-13,14-dihydro-PGD₂ (Δ¹²-PGJ₂), PGJ₂, 15-deoxy Δ¹²⁻¹⁴-PGJ₂ or 2-cyclopenten-1-one.

25. The method of claim 22 wherein at least one of the heat shock proteins induced is HSP70.

26. The method of claim 22 wherein the human has an infectious disease.

27. The method of claim 22 wherein the human has an immune disorder.

28. The method of 22 wherein the human has cancer.

29. The method of claim 22 wherein the human has an inflammatory disorder.

30. The method of claim 22 wherein the human has an HIV infection, an influenza virus infection, a herpesvirus infection, a hepatitis B virus infection, a hepatitis C virus infection, human T-cell lymphotrophic virus type I, human T-cell lymphotrophic virus type II, lassa fever virus, morbillivirus virus, human respiratory syncytial virus, mumps, pneumovirus, adenovirus, hantavirus, cornavirus, Ebola virus, yellow fever virus, Japanese encephalitis virus, papillomavirues), rhinovirus, enterovirus, hepatitis A virus, poxvirus, rotavirus, rubella virus or rabies virus.
